(19) United States
(12) Patent Application Publication
(10) Pub. No.: US 2004/0166141 A1
(43) Pub. Date: Aug. 26, 2004

(54) Methods and devices for modulating the immune response

(76) Inventors: Anthony Cerami, New York, NY (US); Carla Cerami, New York, NY (US); Cohava Gelber, Hartsdale, NY (US); David Dove, New York, NY (US)

Correspondence Address: Frederick J. Hamble, 712 Kitchawan Rd., Ossining, NY, 10562, US

(21) Appl. No.: 10/783,052

(22) Filed: Feb. 20, 2004

Related U.S. Application Data
(63) Continuation of application No. 09/259,929, filed on Mar. 1, 1999.
Application No. 09/259,929 is a continuation-in-part of application No. 09/033,402, filed on Mar. 2, 1998, now abandoned.

(51) Int. Cl. A61K 39/395; A61F 2/00

(52) U.S. Cl. 424/426; 424/140.1

(57) ABSTRACT

The present invention provides methods and devices for inducing, stimulating, blocking and reducing the immune response of a mammal to an antigen, using an implantable device which exposes the antigen in a controlled fashion to cells of the immune system. The device comprises a porous matrix contained within a perforated, impermeable container. By manipulating the bioavailability of antigen within the device, and the timing of introduction of antigen into the device relative to the time of implantation of the device within the mammal, a robust and long-term response can be induced against an antigen, or an existing or potential immune response can be down regulated or blocked. The methods and devices can be used for therapeutic vaccination, and in non-exposed mammals for prophylactic vaccination. Immunity can be cellular, humoral, or mucosal. Suppression of the immune response is useful for the treatment or prophylaxis of such conditions as allergies, autoimmune disease, and in tolerizing mammals to suppress an immune response to transplant antigens. The device can also be used for harvesting immune cells for later reintroduction into the mammal, and for preparing immune serum and hybridomas.

### CROSS-REFERENCE TO RELATED APPLICATIONS

This application is a Continuation-in-part of Application Ser. No. 09/033,402, filed Mar. 2, 1998.

### FIELD OF THE INVENTION

The present invention relates generally to a method for modulating in a mammal an immune response to an antigen, using an implantable device which exposes the antigen in a controlled fashion to cells of the immune system. By creating an artificial environment mimicking the composition and role of a lymph node, and by manipulating the bioavailability of antigen within the device, a robust response may be induced against an antigen, or an existing immune response may be down regulated.

### BACKGROUND OF THE INVENTION

Induction of an immune response to an antigen and the magnitude of that response depend upon a complex interplay among the antigen, various types of immune cells, and co-stimulatory molecules including cytokines and chemokines. The timing and extent of exposure of the immune cells to the antigen and the co-stimulatory milieu further modulate the immune response. Within the body, these various cell types and additional factors are conveniently brought into proximity in lymphoid tissue such as lymph nodes. Of the numerous cell types involved in the process, antigen-presenting cells, such as macrophages and dendritic cells, transport antigen from the periphery to local, organized lymphoid tissue, process the antigen and present antigenic peptides to T cells as well as secrete co-stimulatory molecules. Thus, if antigen reaches lymph organs in a localized staggered manner, under the optimal concentration gradient and under the appropriate environment comprising co-stimulatory molecules, a response is induced in the draining lymph node.

In this manner, a foreign antigen introduced into the body, such as by means of a vaccination, may or may not result in the development of a desirably-robust immune response. Antigens used for vaccination include attenuated and inactivated bacteria and viruses and their components. The success of vaccination depends in part on the type and quantity of the antigen, the location of the site of immunization, and the status of the immune system at the time of vaccination. Not all antigens are equally immunogenic, and for poorly immunogenic antigens, there are few alternatives available to increase the effectiveness of the immunization. Whereas in experimental animals numerous techniques are available to enhance the development of the immune response, such as conjugating the antigen to a more immunogenic carrier protein or biomolecule (e.g., keyhole limpet hemocyanin), or the use of adjuvants such as Freund's Adjuvant or alum, for human vaccinations such techniques and adjuvants are not available. Thus, numerous diseases that would otherwise be preventable by vaccination before exposure to the infectious agent, or in the case of a therapeutic vaccine, that may induce the development of an effective immune response to an existing disease-causing agent or cell, such as cancer, are not available to the patient.

Sponge implant studies have been performed in mammals to assess the immune cell population attracted to a foreign body, which produce what is called a sterile abscess, and sponges prior to or after implantation have been loaded with antigen to further study the attracted cell population. Vallera et al. (1982, Cancer Research 42:397-404) implanted sponges containing tumor cells in mice to examine the composition of cells attracted over a 16 day period, and found that at an early time, cytotoxic cell precursors were present, and cytotoxicity peaked at day 16. Sponges containing tumor cells implanted in mice that had been previously immunized with tumor cells showed a more rapid appearance of cytotoxic cells in the sponge. In neither case did cells from the spleen, lymph nodes or peritoneum show cytotoxicity, which suggested a highly localized response to the antigen in the sponge.

Jenski et al. (1985, J. Immunol. Methods 85:153-161) used similar conditions to follow recovery of cellular immunity in mice receiving syngeneic bone marrow transplants after to sublethal irradiation by implanting sponges loaded with antigen. Recovery of immunity was determined by measuring cytotoxic T lymphocyte activity of cells recovered in the sponges over time. Zangemeister-Wittke et al. (1989, J. Immunol. 143:379-385) injected a tumor vaccine into sponges implanted in tumor-immune mice, and monitored the generation of a secondary immune response at the sponge site. No accompanying effect was apparent in lymph nodes adjacent to the implanted sponge.

Chen et al. (1994, Cancer Research 54:1065-1070) harvested T-cells from sponge implants loaded with irradiated tumor cells to show that these cells could be used to adoptively transfer anti-tumor activity. The frequency of tumor-reactive cytotoxic T cells recovered from the sponge, along with regional lymph nodes and the spleen, were measured in animals receiving the tumor-cell-loaded sponge implants. The sponges contained a four-fold higher frequency of tumor cell-reactive cytotoxic T cells than the lymph nodes, and 50-fold higher than spleen.

While significant research is underway to address these deficiencies in effective and available vaccines, the drawbacks associated with the therapeutic techniques and materials remain. It would therefore be desirable to develop a technique or modality that would overcome the problems presently associated with contemporary immunotherapy and like treatment protocols, such as prophylactic and therapeutic vaccination and to thereby facilitate the development of new, effective and efficient strategies for improved effectiveness of immunization of both healthy mammals and those that may benefit from immunotherapy. Furthermore, the ability to suppress the immune response to a particular antigen or series of antigens offers advantages in treatment of allergies and prevention of transplant rejection, for example. It is accordingly toward the achievement of these and like objectives that the present invention is directed.

### SUMMARY OF THE INVENTION

In accordance with the present invention, a method for modulating the immune response in a mammal to an antigen is provided by implanting within the body of the mammal a device comprising a porous matrix contained within a perforated but otherwise impermeable container. The antigen to which an immune response is desired is present in the porous matrix of the device. The antigen may be present in the device before implantation or introduced after implantation; antigen present in the matrix of the device at the time of implantation may be provided in a non-bioavailable form which becomes bioavailable following implantation. The device will attract cells of the immune system to encounter the antigen within the device, and the encounter will modulate the immune response to the antigen. The perforated container acts as a diffusion barrier, maintaining within the device high levels of cytokines and other co-stimulatory factors produced by immune cells within the device which on exposure to other immune cells in the device, enhance the development of the desired immune response. The perforations permit the ingress and egress of immune cells. The desired immune response may include cellular, mucosal, and humoral immunity, and may be applied to both healthy mammals and those who may benefit from immunotherapy.

For example, in the practice of the invention small amounts of antigen are provided within a device of the present invention prior to implantation within the mammal. Preferably, the antigen is introduced into, or becomes bioavailable within, the porous matrix of the device about three days after implantation within the mammal. A robust immune response to the antigen will be induced in the mammal.

In a further embodiment, the device is composed of biodegradable materials which are eventually degraded within the body. Alternatively, the device may be removed from the body after it has achieved its desired effect.

In its broadest aspect, the present invention extends to an implantable device for immunizing a mammal having the following characteristics: a porous matrix contained within a perforated but otherwise impermeable container, the device further comprising an antigen which is either present within the matrix in a bioavailable form prior to implantation or becomes bioavailable within the matrix after implantation, or the antigen is introduced therein after implantation. The bioavailability of the antigen within the device may be controlled by providing the antigen in a non-bioavailable form such as in a delayed release formulation such as that provided by microspheres, microcapsules, or liposomes, which upon degradation release the antigen into the matrix of the device.

In a further embodiment, the method and device of the present invention can be used to decrease or down regulate the immune response to an antigen within a mammal, by using a high concentration of a particular antigen within the device which results in suppression of the immune response to the particular antigen, and the inhibition of the development of an immune response to the antigen. Cytokines or other co-stimulatory molecules can also be provided within the device.

Further utilities of the methods and devices of the present invention include the harvesting of immune cells from the device for subsequent reintroduction into the body for the purpose of providing adoptive immunotherapy, active immunization, and reconstituting the immune system. Additional uses include improvements in the preparation of polyclonal antibodies (immune serum) and monoclonal antibodies, including the preparation of human monoclonal antibodies in animals harboring human immune cells.

As will be evident below, using the invention described herein, by creating an artificial environment mimicking the composition and role of a lymph node, and by manipulating the bioavailability of antigen within the device, the development of a robust response against an antigen is achieved. Under different conditions for particular antigens, an existing immune response can be down regulated.

### BRIEF DESCRIPTION OF THE DRAWINGS

FIG. 1 shows a time course of the cell population within the device of the present invention over a 9 day period after implantation under the skin of a mammal.

FIG. 2 depicts an analysis of the numbers and types of cells present in the device four days after implantation without any antigen present within the device.

FIG. 3 shows the change in population of various cell types within the device at 4, 7, and 10 days after implantation, in the absence of any introduced antigen.

FIG. 4 shows the effect of introduction of antigen into the device on the numbers of CD3-positive cells (T cells), CD80-positive cells (B cells), and CD14-positive cells (macrophages as representative subset of antigen presenting cells) within the device at days 3, 7, and 10 post-implantation. Antigen was introduced into the device on day 4 post-implantation.

FIG. 5 shows an analysis of the change in expression of CD4 (helper T cells) and CD8 (cytotoxic T cells) positive cells in the spleens of mice ten days after implantation of a device containing either none or various amounts of influenza antigen, or immunization in the footpad with antigen plus adjuvant.

FIG. 6 depicts an assay of the proliferative response of spleen cells to influenza antigen from mice that either had been implanted with a device containing antigen, with or without adjuvant, or immunized in the foot pad with antigen plus adjuvant. Spleen T cell proliferation is measured by gamma-interferon production.

FIG. 7 shows the level of gamma-interferon secretion by T-cells isolated from popliteal lymph nodes of mice immunized in the footpad with influenza antigen plus adjuvant.

FIG. 8 shows the proliferative response of spleen T cells as measured by level of gamma-interferon secretion in mice immunized with influenza antigen, with or without adjuvant, using the device of the present invention.

FIG. 9 shows the activation level of spleen T cells as measured by level of gamma-interferon secretion in mice immunized with a range of doses of the antigen ovalbumin, without adjuvant, using the device of the present invention, as compared to the proliferative response of spleen cells from animals immunized by footpad injection of the same range of doses of antigen with adjuvant.

FIG. 10 also depicts the proliferative response of spleen T cells as measured by level of gamma-interferon secretion in mice immunized with either none, 50 μg, 50 ng, or 50 pg ovalbumin, without or with BCG adjuvant, using the device of the present invention, as compared to the proliferative response of spleen cells from animals immunized by footpad injection of the same doses of antigen with Ribi adjuvant.

FIG. 11 shows the specific anti-antigen IgG2a antibody response in animals implanted with devices containing various doses of HIV gp120 peptide antigen, compared with animals immunized by foot-pad injection with the same doses of antigen plus adjuvant.

FIG. 12 shows the same experiment as depicted in FIG. 11 but following a boost, three weeks after the first immunization, with 10% of the amount of the initial immunization given by the same route. Devices implanted in animals were reloaded with antigen.

FIG. 13 compares the development of specific IgG2a antibodies against the HIV gp120 peptide in animals immunized either with the device of the present invention, with or without Ribi or BCG adjuvant, and conventional footpad immunization with the same adjuvants.

FIG. 14 depicts the specific IgG2a antibody level in animals immunized with various doses of cytochrome C antigen, either using the device of the present invention without adjuvant, or by conventional footpad immunization with the Ribi adjuvant. Circulating IgG2a levels were detected using serial dilutions of the mouse sera.

FIG. 15 shows the level of total Influenza virus-specific IgG developed after immunization of mice with either 0.1 μg or 10 μg of influenza hemagglutinin protein fragment (BHA) in the device of the present invention, without adjuvant, compared with the same antigen dose given in the footpad, with Ribi adjuvant.

FIG. 16 depicts the results of a T cell activation assay using gamma-interferon as the readout. Mice were immunized with influenza antigen using the device of the present invention, without adjuvant, compared with footpad immunization using antigen with adjuvant, and also compared to immunization with antigen in an implanted porous polymer matrix (but not present within the perforated container, as is the device of the present invention).

FIG. 17 shows the results of the above experiment upon in-vitro challenge of spleen cells, from influenza immunized mice, with unrelated antigen (EBV), indicating the specificity of the response developed following immunization using the device of the present invention.

FIG. 18 depicts the results of a protein (bovine serum albumin) diffusion test from sponge alone as compared to the device of the present invention.

FIG. 19 depicts the phenotype of cells extracted from a device of the present invention, without or with influenza antigen, 4 days after insertion into mice. Values for the same cell types in peripheral blood lymphocytes from naive mice are shown.

FIG. 20 shows the levels of stimulatory cytokines in a device of the present invention in the absence of antigen as compared to levels in serum.

FIG. 21 depicts the extent of cytotoxic T cell elicitation, as measured by influenza-infected target cell lysis, by the implanted device of the present invention containing influenza virus vaccine, as compared with animals subcutaneously immunized with influenza virus vaccine together with adjuvant.

FIG. 22 compares the humoral response as measured by the level of influenza virus-specific IgG1 in mice immunized with the device of the present invention containing influenza virus vaccine, with mice immunized subcutaneously with the vaccine plus adjuvant.

FIG. 23 shows the affinity of influenza-specific IgG1 raised in animals immunized with the device of the present invention containing influenza virus vaccine as compared with animals immunized subcutaneously with the vaccine plus adjuvant.

FIG. 24 depicts the survival of mice challenged with influenza virus after immunization with the device of the present invention containing influenza virus vaccine as compared with mice immunized subcutaneously with the vaccine plus adjuvant.

FIG. 25 depicts the titer of influenza-specific human IgG antibodies raised in SCID mice using a device of the present invention, compared with intramuscular immunization.

FIG. 26 shows the affinity of the serum antibodies to influenza antigen (as described in FIG. 25) compared to antibodies raised by intramuscular administration of antigen.

FIG. 27 shows the suppression of the immune response by high amounts of antigen present in a device of the present invention.

FIG. 28 demonstrates the utility of the device of the present invention for immunization with plasmid DNA.

FIGS. 29-31 demonstrate the utility of the device of the present invention for generating an immune response to a polysaccharide antigen.

FIG. 32 depicts the utility of the device of the present invention for eliciting an immune response to a highly conserved and thus poorly immunogenic antigen, cyclophilin.

FIG. 33 shows the utility of the device of the present invention for eliciting an immune response to a parasite antigen using whole animals.

### DETAILED DESCRIPTION

The present invention provides a method for modulating in a mammal an immune response to an antigen, using an implantable device which exposes the antigen in a controlled fashion to cells of the immune system. By creating an artificial environment mimicking the composition and role of a lymph node, and by manipulating the bioavailability of antigen within the device, a robust response can be induced against an antigen, or an existing immune response can be down regulated. The device comprises a porous matrix, i.e., a sponge-like material, surrounded by or contained within a perforated but otherwise impermeable coating or barrier, herein referred to as a container. The perforated container acts as a diffusion barrier to maintain high concentrations of immune cell secretory products within the device. The antigen to which an immune response is to be raised or down regulated is present within the porous matrix of the device, either before or after the device is inserted under the skin. For inducing a robust immune response, the antigen is preferably bioavailable within the matrix of the device about three days after the device is inserted. This can be achieved by injecting the antigen into the device around this time, or by using a device wherein the matrix comprises a delayed release form of the antigen, which after about three days becomes bioavailable. For down regulating or suppressing the immune response to a particular antigen, and depending on the particular antigen, the fully bioavailable antigen is preferably provided within the device before it is implanted within the mammal. In the instance where the antigen is placed within the device after the device has been implanted, the antigen can be introduced into the device transdermally using a syringe and hypodermic needle, by identifying the location of the device, and inserting the needle into the device. The device can be left in place or removed from the body. It can be made of biodegradable materials which will eventually disintegrate within the body, or it can be removed after it has achieved its desired effect. Antigen can be reintroduced into a device at a later time. The antigen can be used within the device alone or with an adjuvant, or a combination of adjuvants. Preferably, no adjuvant is used.

The present invention offers improvements to many forms of immunotherapy, wherein a desirable immune response is developed or increased against an antigen, and contrarily, wherein an existing immune response or the potential to develop an immune response against a particular antigen can be suppressed or blocked, respectively. These include vaccination such as vaccinating healthy mammals against particular antigens, and therapeutic vaccination procedures. Blockage or suppression of the immune response can be applied to a mammal before encountering the particular antigen, such as a future transplant recipient, a mammal anticipated to be exposed to an allergen, or one predisposed to develop an immune response to an exogenous or endogenous antigen, such as in autoimmune diseases. Suppression of an immune response can be useful after exposure to the antigen, such as a mammal with an allergic or anaphylactic response to an antigen, or one undergoing rejection of a transplant. The methods and devices of the present invention are directed toward all forms of immunity including cellular, humoral, and mucosal immunity.

The configuration of the device and its method of use simulate the structure and function of mammalian lymphoid tissue, and particularly a lymph node. In the body, introduced foreign antigens are taken up and processed by antigen presenting cells, macrophages and dendritic cells, which enter the lymph nodes and present immunogenic peptides derived from the antigen in a particular conformation with MHC antigens to T lymphocytes. A special subset of T cells (CD4-Th2) provides help for B-cells and supports the development of high affinity humoral response. B cells can interact with antigen directly (especially multiple-unit antigens or recall antigens) and differentiate into plasma cells secreting antibodies specific to the immunizing antigen. The antigen presenting cells also release cytokines, lymphokines, and chemokines which co-participate in the development of the immune response. The perforated container component of the device maintains a diffusion barrier which maintains levels of the antigen and immune cell secretory products, such as cytokines, within the device and in proximity of the immune cells within the device. The perforations restrict the diffusion of these molecules from the device but permit the free ingress and egress of immune and other cells into and out of the device. Very small amounts of antigen have been found to be adequate to induce a robust immune response. The interaction between the antigen present within the device and the immune cells and co-stimulatory molecules within the device is thus optimized to enhance the development of the immune response to the antigen, as well as imparting long-term immunity by producing a population of memory cells.

Implantation of the device of the present invention into the mammalian body initiates what is termed a sterile abscess, in which certain cells of the immune system are attracted to the foreign body and enter through the limited number of perforations. Over the first few days, an increasing cellular population accumulates within the device, even in the absence of any antigen. With the bioavailability of antigen in the device at about day three, and the encountering of the antigen by immune system cells present within the matrix, the antigen is taken up and processed by antigen presenting cells and presented to T lymphocytes. As the device has limited exposure to the outside by way of the perforated container, immune cells can enter, but the antigen is retained within the device and its concentration remains high, as do the concentrations of co-stimulatory factors secreted by the cell population with the device, much in the same fashion as within a lymph node. After T lymphocytes become primed and activated, they can leave the device through the limited number of perforations and re-enter the circulation. Thus, the device provides a means for controlling the exposure of the antigen to immune system cells and maintaining levels of the cytokines and other factors necessary for developing a robust immune response. As will be seen in the examples below, the device provides a several orders of magnitude improvement in the development of an immune response to a particular antigen.

In a further theoretical consideration the implantation of a device of the present invention containing a large amount of a particular antigen can function in an opposite manner as described above, in that it can down regulate or suppress an existing or potential immune response to the antigen. Exposure of T cells to the excess antigen apparently initiates apoptosis of antigen-specific T cells, thus eliminating antigen-specific T cells and progenitors, effectively suppressing the cellular response and also the humoral response directed towards the antigen. Hyperactivation of T cells mediated by addition of cytokines (such as IL-2, IL-4, γ-IFN, IL-12) to the antigenic stimulus or exposing the T cells to additional antigenic stimulus when in a refractory state (i.e. before activation state subsides) will trigger apoptosis of cells and deletion of the reactive clones (B or T cells) from the antigen-specific repertoire. The selection of the appropriate concentration of a particular antigen for achieving the stimulation or the suppression of the immune response using a device of the present invention will be known or readily determinable by the skilled artisan. The immunogenicity of a particular antigen and therefore the ranges of its immunosuppressive and immunostimulatory doses of a particular antigen can be determined by in vitro or in vivo methods known in the art.

Moreover, the improved immune response achieved by using the device of the present invention exceeds that achievable with traditional immunization methods which usually, in the case of animals, includes the use of adjuvants. On a theoretical basis, the controlled exposure of the antigen alone to immune cells and their co-stimulatory factors within the device appears to provide an optimal environment for achieving a robust immune response, which is superior to that achievable by the use of adjuvants.

Immunization offers an effective method for prophylaxis against a number of infectious disease agents, such as viruses: for example, influenza, HIV, papilloma, hepatitis, cytomegalovirus, polio, and rabies; bacteria, for example E. coli, Pseudomonas, Shigella, syphilis, mycobacteria, Chlamydia, rickettsiae, and Serratia marcescens; fungi, for example, Aspergillus and Candida; and protozoan and multicellular parasites, for example, Schistosoma, Plasmodium, Onchocerca, and ameobae. Immunization also offers the potential using a therapeutic vaccine to treat an individual already suffering from an infectious disease. Furthermore, noninfectious diseases may be prevented or treated through immunization, such as cancer. Many antigens, however, are poorly immunogenic, and immunization has not proved to be an appropriate strategy for the prevention or treatment of these diseases. Adjuvants as are used routinely with animal immunizations are not available to increase the immune response. In addition, the immune system of an individual or patient may not be functioning optimally to be able to produce an immune response to an antigen that would otherwise produce a robust immune response in an otherwise healthy individual. These situations offer the opportunity for the device of the present invention to stimulate a robust immune response where such a response would not be possible by conventional immunization procedures.

Suppression of the immune response may also be desirable to treat certain conditions, such as allergies, or to prepare patients for the exposure to foreign antigens, such as for transplant. Inappropriate immune responses are believed to be the underlying etiology in a number of autoimmune and other diseases, such as type I diabetes, rheumatoid arthritis, multiple sclerosis, uveitis, systemic lupus erythematosus, myasthenia gravis, and Graves' disease. By implanting in an individual a device of the present invention containing the suspect antigen, entry of cells primed to recognize the antigen can be induced to undergo apoptosis, and be eliminated from the immune system. Elimination of progenitor antigen-specific cells can permit the later transplant of foreign antigens without rejection.

Further utilities of the present invention include improvements in the generation of polyclonal antibodies (immune serum) and monoclonal antibodies in laboratory animals and obtaining the desired isotype of antibody so generated. In one embodiment, a procedure for preparing polyclonal (immune serum) and monoclonal antibodies against an antigen available only in minute quantities can be performed by the device of the present invention. The device can be provided with a small amount of the rare antigen, in order to immunize the animal, after which spleen cells can be harvested. This procedure offers an improvement over current tedious and unpredictable method of introducing the rare antigen directly into the spleen. Furthermore, the need for a boost immunization can be obviated by use of the device of the present invention, and, in addition, an immune response will be generated more quickly. A shortened time required to immunize animals will allow the generation of monoclonal antibodies more rapidly. In another embodiment, immune cells for the production of hybridomas can be harvested from the device after immunization of an animal with an antigen provided within the device. This procedure can also be used to generate human monoclonal antibodies, by implanting a device of the present invention into an individual, loading the device with antigen, and then harvesting immune cells from the device for the production of hybridomas. The above-mentioned polyclonal antibodies (immune serum) and monoclonal antibodies can be used for diagnosis, basic research, imaging and/or therapy. In another embodiment, human monoclonal antibodies can be generated using the device of the present invention implanted in a severe combined immunodeficiency (SCID) mouse, by the following procedure. First, human peripheral blood lymphocytes are injected into a SCID mouse, wherein the human lymphocytes populate the murine immune system. After implantation of a device of the present invention comprising the desired antigen which is bioavailable at about three days after implantation, subsequent harvesting of cells from the device will provide human B lymphocytes cells which can then be used to prepare hybridomas which secrete human antibodies against the desired antigen.

A further utility of the device of the present invention is in collection of immune cells from a mammal for later reintroduction into the mammal. Cells can be removed from the device, for example, by aspiration from the implanted device or collection from the device after removal from the body by dissolving the polymer matrix, subsequent storage of the cells, for example by cryopreservation, and reintroduction into the mammal at a later time. This can be particularly useful for mammals undergoing whole-body radiation therapy. A device of the present invention, without containing antigen, can be implanted and maintained for seven to ten days, and subsequently the device or its contents removed and the cells contained therein cryopreserved. Following radiation therapy, the mammal can have the cells reintroduced into the body, whereby the cells will reconstitute the immune system. In another embodiment of this utility, co-stimulatory factors such as cytokines which induce the proliferation of immune cells can be introduced into the device to increase the yield of cells within the device, before harvesting. In a further embodiment, immune cells collected from a device provided with antigen can be used for active immunization, wherein the cells can be stored and then reintroduced into the mammal after, for example, a course of chemotherapy or other therapeutic manipulation. In a still further embodiment, cells collected from a device can be cryopreserved, and at a later time be exposed to the antigen (for example, a cancer antigen) for ex-vivo propagation of T cells prior to introduction into the body, for adoptive immunotherapy.

In another utility of the present invention, the device can be used to transfect immune cells within the device with genes. The transfected immune cells can then prime immune cells within device, and/or after migration out of the device, can prime immune cells in distal organs. For example, DNA, RNA, or cDNA encoding a tumor-specific antigen or viral, bacterial, or parasitic antigen can be placed within the device, with or without the corresponding protein antigen. Antigen-presenting cells entering the device can be transfected with the gene, subsequently expressing the antigen protein and migrating to peripheral sites within the body where they will stimulate immune cells.

The device of the present invention can be manufactured by methods known to the skilled artisan, and the size and shape can be varied, as long as its configuration enables it to function in the manner described herein. As described above, the device provides a diffusion barrier for small molecules, such as the antigen introduced into the device, and cytokines and other factors secreted by immune cells within the device, but permits the ingress and egress of immune cells into and out of the device. Thus, passive diffusion of proteins and other small molecules is limited, but immune cells undergo active movement into and out of the device. In one embodiment, a device for convenient insertion into a small incision in the skin and later removal, if required, can be fashioned from a short segment of hollow, biologically-inert plastic tubing, such as silicone tubing. The porous polymer matrix, i.e., a sponge-like material, is fitted into the bore of the tubing. The open ends may or may not be sealed. A small number of perforations are made into the walls of the tubing. Variations in the number, shape and size of the perforations are within the scope of the invention. The antigen can be introduced in the form of a solution or suspension of antigen or a non-bioavailable form of antigen, by incorporation into the matrix during manufacture, or injection into the matrix, either through one end of the tubing or through the wall of the tubing itself.

The antigen can be provided in the matrix of the device directly, referred to herein as the bioavailable or fully bioavailable form, or it can be provided in a non-bioavailable form, which subsequently becomes bioavailable. Incorporation of the antigen into a formulation that provides controlled-release, sustained-release, or delayed-release characteristics is suitable; such processes and formulations can include microencapsulation, liposomes, and microspheres. Preferably, for the purpose of stimulating or increasing the immune response to the antigen, the formulated antigen becomes bioavailable within the matrix of the device about three days after implantation. Suitable release formulations are known in the art.

In another embodiment, a device can be prepared from a polymeric matrix material in the desired shape of the final device, and an impervious coating applied to the surface. Perforation can be made subsequently. Alternatively, a polymer matrix of a certain porosity and degree of crosslinking can be extruded in the desired shape of the device, and then the exterior treated with an agent to further cross-link the polymer at the surface of the device to effectively form an impermeable coating or container. The coating subsequently can be perforated and the antigen introduced. Increased polymerization of the surface of the device can also be effected by surface ultraviolet treatment if an ultraviolet light-curable polymer is used. The examples provided herein are not intended to be limiting, as the skilled artisan will be facile in the design of a suitable device with the aforementioned properties.

Materials comprising the device can be selected from a wide range of suitable naturally-occurring or synthetic compositions. In one embodiment, the polymer matrix can be a biologically compatible material such as hydroxylated polyvinyl acetate. Another matrix is polyurethane, which is widely available. Other suitable materials include ethylene/vinyl acetate copolymer, polylactic acid, polyglycolic acid, polylactide-glycolide copolymer, collagen, cross-linked collagen, and gelatin.

As described above, achieving a perforated but otherwise impermeable barrier or coating around the polymer matrix can be achieved by any number of means. In one embodiment, segments of polymer matrix are placed within the lumen of a short segment of biologically compatible plastic tubing, such as silicone tubing. In one example, a 2.5-cm segment of tubing with internal diameter of 0.15 cm and an outer diameter of 0.2 cm was fitted with a corresponding 2.5-cm segment of pre-wetted hydroxylated polyvinyl acetate matrix. In other examples, the barrier or coating can be made of a suitable naturally-occurring or synthetic material, such as a plastic or other polymer, such as polyethylene, cross-linked collagen, polyethylene, silicone, latex resin, polystyrene, acrylic resin, polyvinylpyrrolidone, and combinations of these materials. One commercially available material is SILASTIC® silicone tubing from Dow Corning. These non-limiting examples are simply exemplary of the range of suitable compositions useful for the present invention.

As a non-limiting example of the preparation of a device of the present invention, and in keeping with the aforementioned desired characteristics of the device which restricts the diffusion of small molecules from the device but permits the ingress and egress of immune cells, a device can be prepared manually as follows. A 1.125 inch length of silicone tubing of an outer diameter of 0.047 inches and a radius of 0.0235 inch can be used as the impervious container of the device of the present invention. Holes can be manually punched with a 20 gauge hypodermic needle through the wall of the device, which produces holes of approximately 1/16 and 1/32 of an inch in diameter. Twenty holes were punched in the tubing. These parameters serve only as a guide to the characteristics of the device, and a skilled artisan will be aware of other parameters which will achieve the same objectives as set forth above, that is, to provide unrestricted cellular ingress and egress but to restrict and confine the diffusion of small molecules, such as cytokines, within the device.

A biodegradable device of the present invention can comprise a matrix and container of materials known to slowly degrade within the body. Such materials include gelatin, collagen, cross-linked collagen, polylactic acid, polylactide-glycolide copolymer, and other materials known to the skilled artisan. Thus, a preferred fully biodegradable device for stimulating the immune response may comprise a biodegradable container, a biodegradable matrix, and a biodegradable, delayed release formulation of antigen contained within the matrix. The latter formulation releases the antigen at about three days after implantation; the matrix and the container begin to significantly biodegrade after the useful life of the device, about 10 days after implantation.

The perforations in the container of the device may be introduced by any of a number of methods, including manual and automated procedures. A small number of perforations are optimal, preferably around 10 per centimeter of tubing, although this will depend somewhat on the size and shape of the device. In accordance with the theoretical considerations provided above, the role of the perforations is to permit entry of immune system cells into the device which then come in contact with the antigen and co-stimulatory molecules to become primed, and then to permit egress of the primed cells. These objectives must be achieved while at the same time the perforated device must contain and maintain the desired levels of the antigen and co-stimulatory factors such as cytokines produced by the immune system cells within the device. A suitable number and size of perforations will achieve these needs, as illustrated in the examples below. In the instance where the device is prepared from a segment of tubing, the ends of the tubing can be left open to act as perforations, as well as a receptacle for the introduction of a needle or other means for loading antigen, either prior to implantation or subsequent to implantation.

The device can be implanted at a suitable site on the body where ease of insertion, antigen loading, and removal can be accomplished with minimal discomfort to the patient. One suitable site is the medial surface of the upper arm. In another embodiment, the device is made of biodegradable materials, insofar as the device will degrade after its useful life and not need to be removed.

For stimulating an immune response bioavailable antigen may be present within the device at the time of implantation or preferably afterward; if afterward, the timing is preferably about three days after implantation. A non-bioavailable formulation of antigen with delayed release characteristics may be present in the device at the time of implantation which releases antigen into the matrix of the device about three days after implantation. After about three days, a sufficient number and types of cells able to respond to the presence of antigen are present within the device, along with levels of cytokines and other co-stimulatory molecules secreted from these cells and maintained within the device as a result of the diffusion barrier afforded by the perforated container, and the subsequent introduction of antigen into the device initiates the development of an optimal immune response.

The device, if not constructed of biodegradable materials as described above, can be removed by a simple surgical procedure after it has achieved its desired function. Generally, after about 10 days, the immune cell population has egressed from the device and it is no longer functional. On the other hand, the device can be refilled with antigen at a later date in order to boost the immune response.

As described above, to achieve the desired stimulation or suppression of the immune response, the timing of the bioavailability of the antigen within the matrix of the device is important, and is dependent on the characteristics of the particular antigen. In general, a large concentration of fully bioavailable antigen present at the time of implantation will have the effect of suppressing the immune response to the antigen. Also, in general, a small amount of antigen fully bioavailable about three days after implantation of the device will have the effect of stimulating the immune response. These conditions may be varied without detracting from the utility of the invention, depending on the characteristics of the particular antigen to be used in the device. The skilled artisan will be able to assess the immunogenicity of the particular antigen, by standard in vitro or in vivo methods, and determine the appropriate concentration of antigen to achieve the desired effect.

The method of use of the device of the present invention for enhancing or stimulating the immune response to an antigen is intended to yield a population of immune cells, T and B lymphocytes, that will mount an effective immune response against the antigen used in the device.

It is another objective of the present invention to down regulate the immune response against a specific antigen using the aforementioned method and device. By incorporating high doses of an antigen in the device immune cells entering the device and encountering the antigen may be induced to undergo apoptosis. Conditions such as graft and transplant rejection may be preventable or treatable if the recipient, prior to of after transplant, is provided a device of the present invention containing donor antigen. General conditions that are amenable to a down-regulation of the immune response include: transplantation, in which it is desirable to tolerize the recipient with donor blood cells to down regulate the immune response of the recipient to the donor's graft; autoimmune diseases, in which tolerization to autoantigens is desirable to down regulate pathogenic T cells and ameliorate the formation of immune complexes with endogenous antigens, such as collagen in rheumatoid arthritis; diabetes, in which it is desirable to tolerize diabetic patients to insulin or GAD; and myasthenia gravis, in which patients can be tolerized to avoid an immune response against the acetylcholine receptor. Furthermore, the immune response which characterizes allergies and allergic reactions can be treated by tolerizing or desensitizing the patient to the allergen by inducing apoptosis in the immune cells responsible for the immune response. Examples of allergies and antigens that can be utilized in the device of the present invention to suppress the immune response include the cat allergy allergen DERP-1, and poison ivy/oak allergies caused by urushiol-modified peptides.

The following examples are presented in order to more fully illustrate the preferred embodiments of the invention. They should in no way be construed, however, as limiting the broad scope of the invention.

### EXAMPLE 1

An example of the device of the present invention was prepared using a 2.5-cm length of silicone tubing with an internal diameter of 0.15 cm and outer diameter of 0.2 cm, fitted with 2.5 cm-long segment of hydroxylated polyvinyl acetate sponge. The device was immersed in a container containing phosphate-buffered saline and autoclaved for sterilization. Female BALB/c mice (6-8 weeks old) were anesthetized with Avertin. The device was inserted through a 0.5-cm dorsal midline incision on day 1. In some animals, 50 μl of 1 mg/ml influenza antigen (FLUSHIELD® influenza virus vaccine, trivalent, Types A & B; obtained from Henry Schein®, Melville N.Y.) was introduced into the device on day 2 post-implantation, by inserting a hypodermic needle through the skin near the implanted device and then guiding by feel the needle into one end of the device.

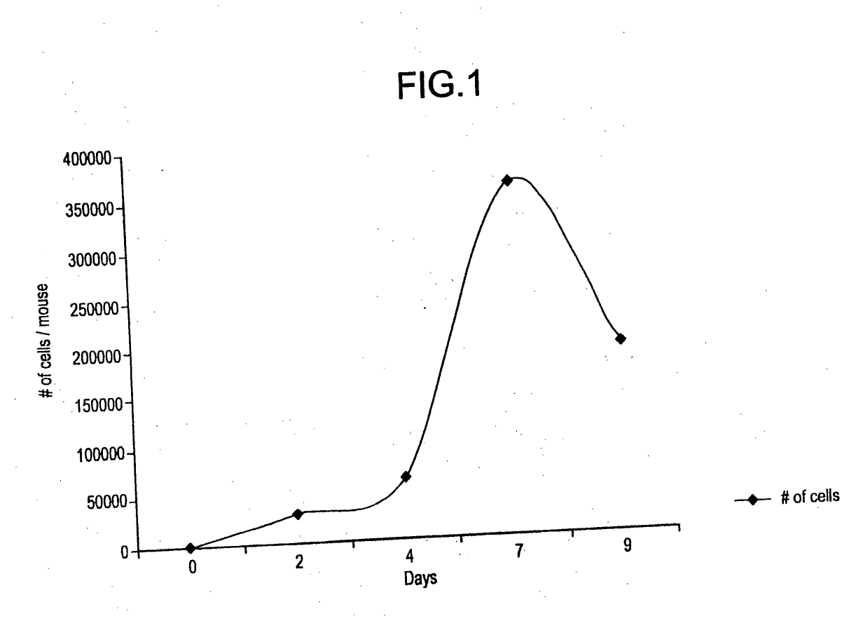

Using a hypodermic needle, fluid was aspirated from the implanted devices from each of five animals on days 2, 4, 7, and 9 post-implantation, and then cells in the fluids were combined, washed, and counted. As shown in FIG. 1, in animals implanted with devices not provided with antigen, the cell population began to rise on day 4 and peaked on day 7. By day 9, the decreasing number of cells in the device indicates that cells that had migrated into the device were migrating out of the device and into the periphery. When influenza antigen was added to the device on day 2 post-implantation, the number of cells present in the device on day 4 post-implantation was several-fold higher, 225,000, as compared to 60,000 cells in devices without antigen. Thus, the presence of antigen within the device augmented the recruitment of cells into the device and/or triggered proliferation of cells within the device.

### EXAMPLE 2

The numbers and phenotypes of cells present in the device without added antigen were determined in this experiment, using BALB/c mice. At four days post-implantation of a device as described in Example 1, the cells from each of five animals were aspirated from the devices, combined, washed and divided into several tubes. Fluorescein isothiocyanate- or phycoerythrin-labeled monoclonal antibodies specific to the following markers (CD14, CD45/B220, CD11b, CD40, CD11c, CD80, CD86, CD62P, CD62E, CD3, and I-Ad) were added, and after incubation for 30-45 minutes at 4° C., the cells were washed and the geometric means of the fluorescence determined by flow cytometry. For comparison, the fluorescence of peripheral blood lymphocytes from naive, syngeneic mice using the same array of specific antibodies was determined, and the results for each antibody expressed as the percent increase in fluorescence of the cells from the device over that of peripheral blood lymphocytes.

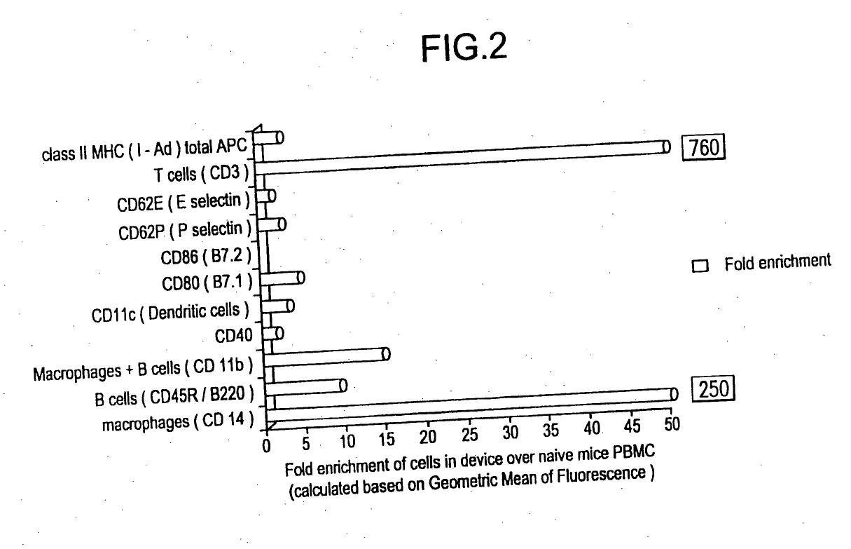

FIG. 2 presents the phenotype of cells extracted from device 4 days post-insertion. Migration and accumulation of T cells (CD3, CD40), macrophages (CD14, CD11b, class II MHC, CD80), dendritic cells (CD40, CD11c, Class II MHC, CD80) and B cells (Class II MHC, CD45/B220, CD11b) in high density were evident in the device.

### EXAMPLE 3

The changes in the cell population within the device described in Example 1 over time was evaluated by harvesting cells from the device at days 3, 7, and 10 after implantation. In the first experiment, no antigen was provided within the device. Cells aspirated from devices implanted in five mice at each time point were combined, and the density of cells bearing CD3, CD8, CD80, CD44, CD11c, CD45R/B220, and CD14 markers were determined following the same procedure described in Example 2, expressing the values as percent increase over peripheral blood lymphocytes of naive mice.

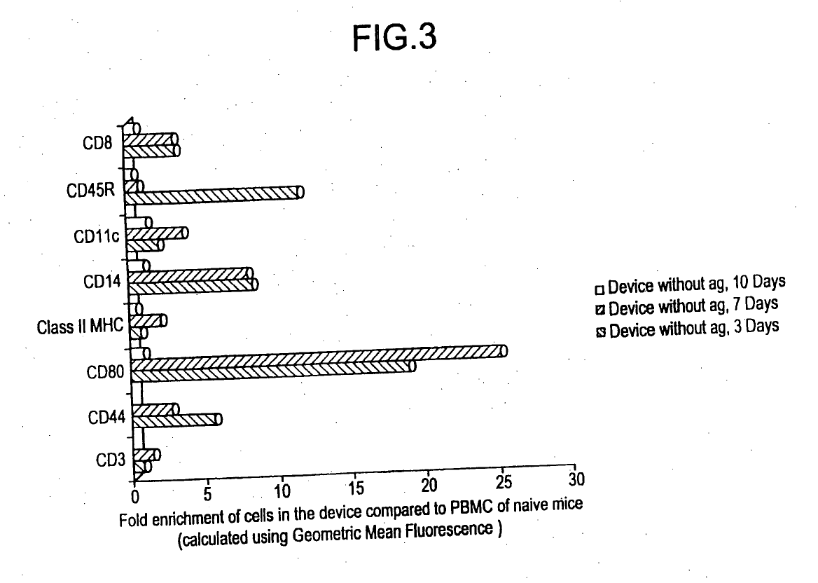

FIG. 3 shows the density of the enumerated cell phenotypes at days 3, 7, and 10 post implantation. For all markers, enrichment of the cell type in the device was apparent at days 3 and 7, and by day 10, the population had migrated out of the device.

### EXAMPLE 4

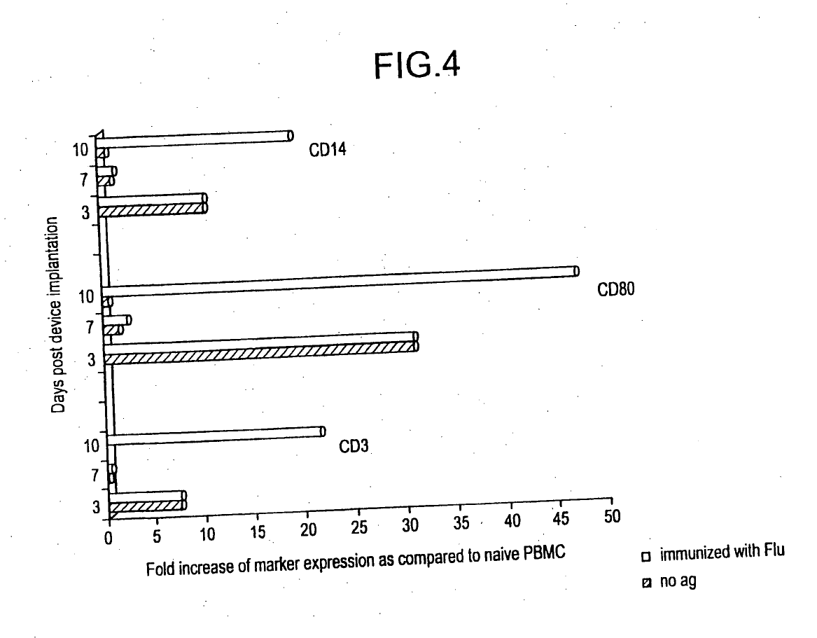

In a second experiment set up exactly like the above, influenza antigen was provided within the device at day 2 post-implantation. As shown in FIG. 4, the density of T cells and antigen presenting cells (CD3, CD80 or CD14) was further augmented by the presence of antigen in the device, and furthermore, resulted in persistence of these three cell types at day 10. The addition of antigen may have retarded the departure of the immune cells from the device.

### EXAMPLE 5

The distal effect of implantation of the device provided with influenza antigen was evaluated by measuring the change in expression of CD4 and CD8 T cells in the spleens of implanted animals. Methods as described above were followed, and the T-cell phenotypes were determined by the method of Example 2. The devices were provided with 0, 5, 10 or 50 μg influenza vaccine 2 days post-implantation, and spleens harvested from the animals 10 days post-immunization (12 days post-implantation). Spleen cells were isolated by gently teasing the spleens between the rough sides of glass slides. The cells were washed with PBS and red blood cells were depleted by 5 minutes incubation in Red Cell Lysis Buffer (Sigma). CD4 arid CD8 fluorescence was determined by flow cytometry as described above. As a control, a conventional, standard immunization protocol was followed in which mice were injected with the same amount of influenza antigen in the footpad, together with Ribi adjuvant R-700, an emulsion containing monophosphoryl lipid A and synthetic trehalose dicrynomycolate (MPLA+TDM), manufactured by Ribi ImmunoChem Research, Inc. Control mice were also bled 10 days after immunization.

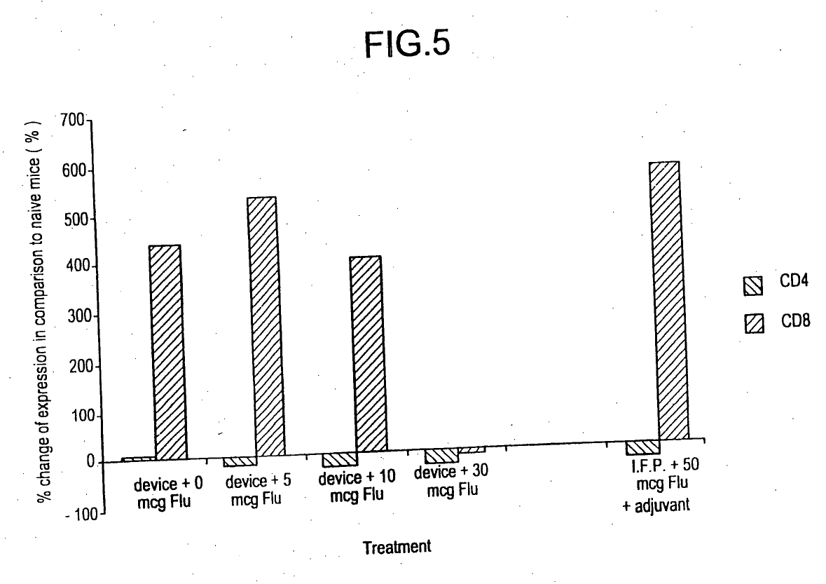

As shown in FIG. 5, T cells derived from spleen cells of mice implanted with the device exhibited increased density of CD4 and CD8 molecules following loading of the device with influenza antigen. The levels of cytotoxic T cells (CD8) in the spleens from animals immunized by conventional methods with 50 μg antigen plus adjuvant was equivalent to that induced by 10 μg of antigen in the device of the present invention. Furthermore, the device without antigen also showed an expansion of CD8 cells in the spleen, as a result of the sterile inflammation induced by the device. It is noteworthy that in animals implanted with devices of the present invention provided with 50 μg of antigen (the same amount as that inoculated via footpad with adjuvant in the control animals) showed a loss of CD8 cells expression in the spleen. One explanation is that administration of a high dose of antigen into the device resulted in apoptosis of antigen-specific cells. Exertion of the extremely strong stimulus may have proceeded to trigger a ruffle effect and its end result was elimination of the entire CD4+CD8+T cells from spleen of the treated mice. This has application in the desired down regulation of the immune response in instances in which an immune response is deleterious, e.g., in transplant rejection.

The explanation for the increased efficiency of immunization using the device of the present invention is presumed to be that the amount of antigen that actually arrives at the lymph nodes in an animal following injection of the antigen at a peripheral site is many logs less than is given in the immunization. Thus, the optimal dose for immunization using the device of the present invention for evoking a positive immune response is expected to be substantially lower (i.e., several logs lower) than the dose utilized in conventional immunization.

### EXAMPLE 6

The strength of the immune response to influenza antigen provided in the device of the present invention was evaluated using secretion of gamma-interferon by spleen T cells as a marker. Gamma-interferon secretion is one of the principal attributes of Th1-type and CD8 cytotoxic T cell response, known to be the protective arm against intracellular pathogens and cancer. Mice were implanted with devices described above and 5 μg influenza vaccine was provided at 3 days post-implantation. The control group was immunized in the footpad with 50 μg antigen plus Ribi adjuvant. Ten days post-immunization, spleen cells were isolated as in the previous example, plated, and gamma-interferon production measured using an ELISA kit (Endogen) after stimulation with a range of antigen levels (1.4 to 180 μg/ml).

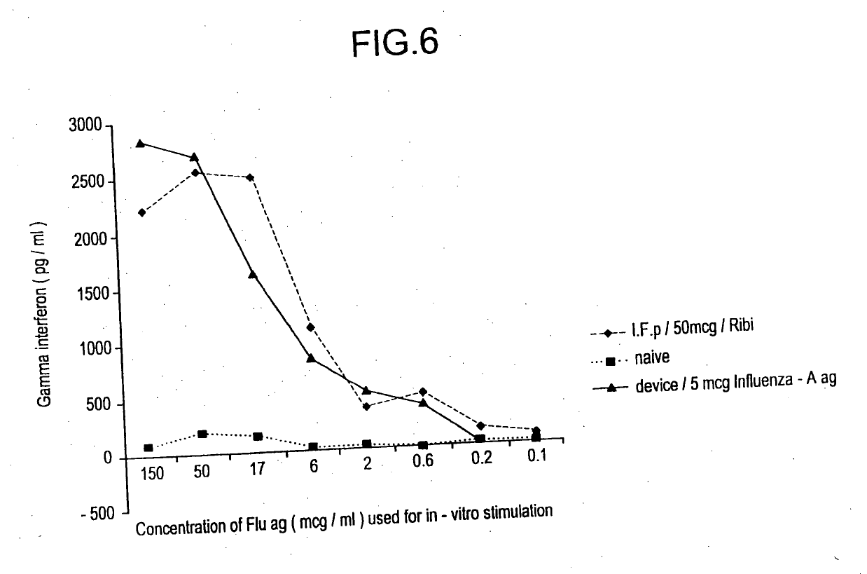

FIG. 6 shows that immunization of the animals with 5 μg antigen in a device of the present invention produced the same level of T-cell activation produced by a 50 μg immunization, with adjuvant, in the footpad. Thus, a strong immune response can be achieved without the use of adjuvant using the device of the present invention, and with a significantly smaller amount of antigen.

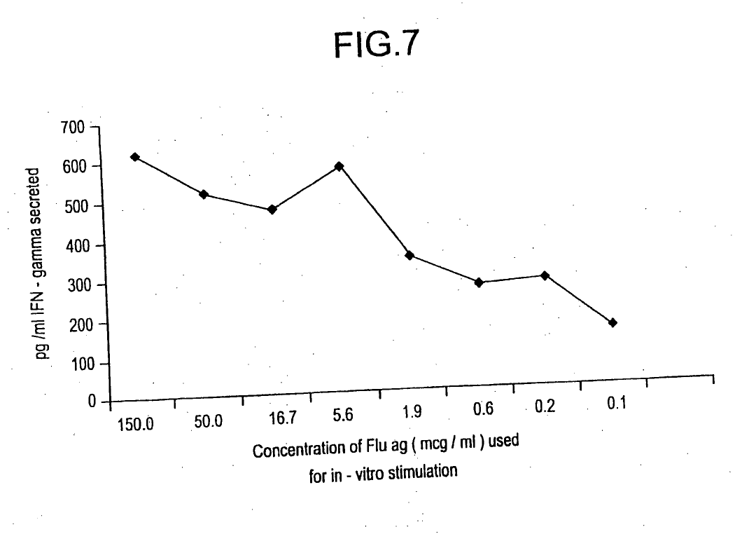

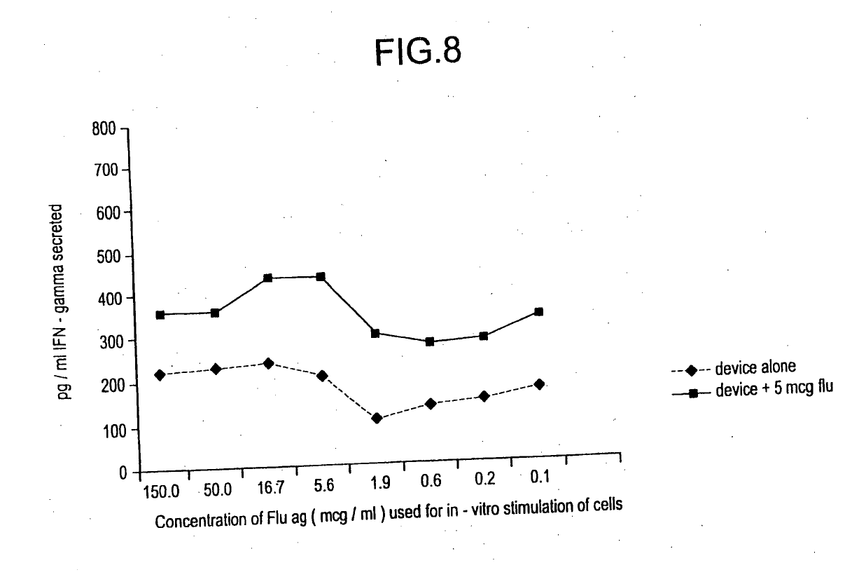

The gamma-interferon secretion from T-cells derived from the popliteal lymph nodes from the same animals was evaluated, after exposure of the isolated T cells to a similar range of antigen concentrations. As shown in FIG. 7, the gamma-interferon secretion by T cells from animals immunized with antigen plus adjuvant in the foot pad was not as robust as that produced from splenic T-cells from animals immunized using the device of the present invention (compare to FIG. 6). As the popliteal lymph nodes are those which drain the footpad area and thus expected to be primed by the presence of antigen, this result further shows the efficacy with which the device of the present invention can be used to produce a robust immune response without the need for adjuvant. In a further evaluation, the cell to population remaining in a device (having been provided with antigen at day 3) 10 days after implantation was assessed for gamma-interferon secretion in response to exposure to antigen. FIG. 8 shows that a very low level of gamma-interferon is secreted in response to an in vitro challenge with influenza antigen in comparison to the level of response recorded with spleen cells. This result implies that the effector cells primed by intra-device immunization migrated out of the site and at day 10 (when the aspirate was taken from the device) most of the effector population was depleted. FIGS. 1 and 3 depict loss of cells with the B and T cell phenotype from device by day 9 and 10, confirming the possibility of their egress to the peripheral organs.

### EXAMPLE 7

In a further experiment aimed at examining specifically the strength of the immune response to the antigen induced by the device of the present invention, following implantation and loading the device with antigen, a spleen cell proliferation assay was performed. The same protocol as described in the previous examples was followed. The antigen in this experiment was ovalbumin; devices were provided with various amounts of ovalbumin ranging from 10 pg to 50 μg; control animals were immunized by footpad injection with the same amount of antigen, plus Ribi adjuvant.

Ten days post-implantation the mice were euthanized and the spleens were removed. A proliferation assay was set up in 96-well plates. Two hundred thousand spleen cells per well were exposed to 180 μg/ml antigen for 72 hours at 37 C; as controls, the same dose of Epstein-Barr virus antigen was used. After exposure to antigen or control, the cells were pulsed for 6 hours with ³H-thymidine, and incorporation of label was measured and expressed as a stimulation index.

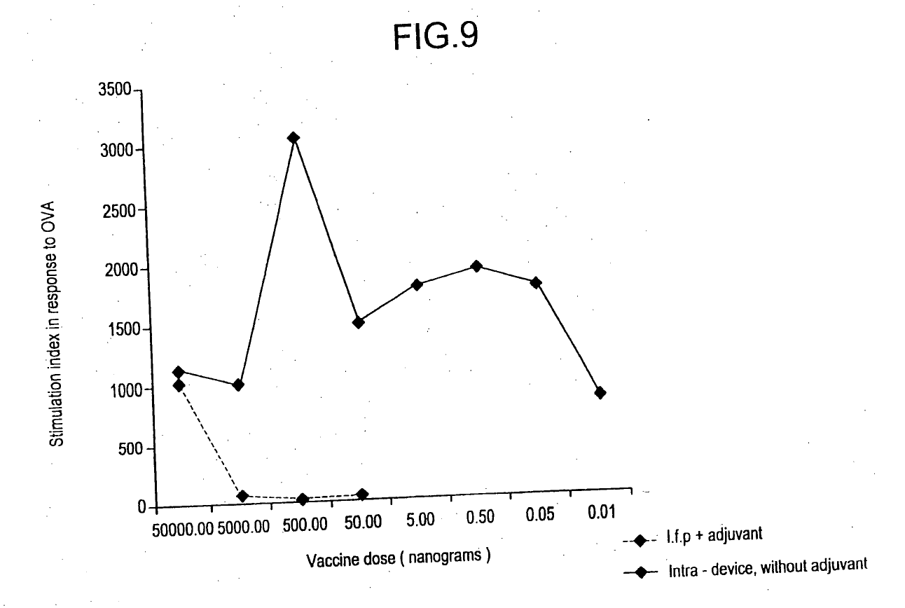

FIG. 9 shows that immunization of mice using the device provided with ovalbumin resulted in a robust antigen-specific spleen cell proliferative response. A response was detected in animals immunized with as low as picograms of antigen. Spleen cells from animals immunized by conventional methods with adjuvant produced a proliferative response only at 5 to 7 logs higher antigen concentration. Negligible levels of stimulation to control (Epstein-Barr virus) antigen (data not shown) indicate that the evoked immune response to ovalbumin was highly specific.

In a further experiment using ovalbumin as antigen, mice were immunized either using the device of the present invention or by footpad, with 50 μg, 50 ng, or 50 pg ovalbumin, either alone in the device, with BCG adjuvant in the device, or via footpad with Ribi adjuvant. The BCG (Bacille Calmette Guerin) adjuvant used in these experiments, TheraCys(R), is a freeze-dried suspension of an attenuated strain of Mycobacterium bovis, prepared by Connaught Laboratories Limited for treatment of carcinoma in situ of the urinary bladder. Ten days after immunization, spleen cells were harvested and exposed to various concentrations of antigen (1.4 to 180 μg/ml) or to the same range of a control antigen, Epstein-Barr virus, in vitro. After a 72-hour incubation, the cells were pulsed with ³H-thymidine for 6 hours, and the incorporation of label expressed as a Stimulation Index and used as a measure of T cell stimulation and proliferation.

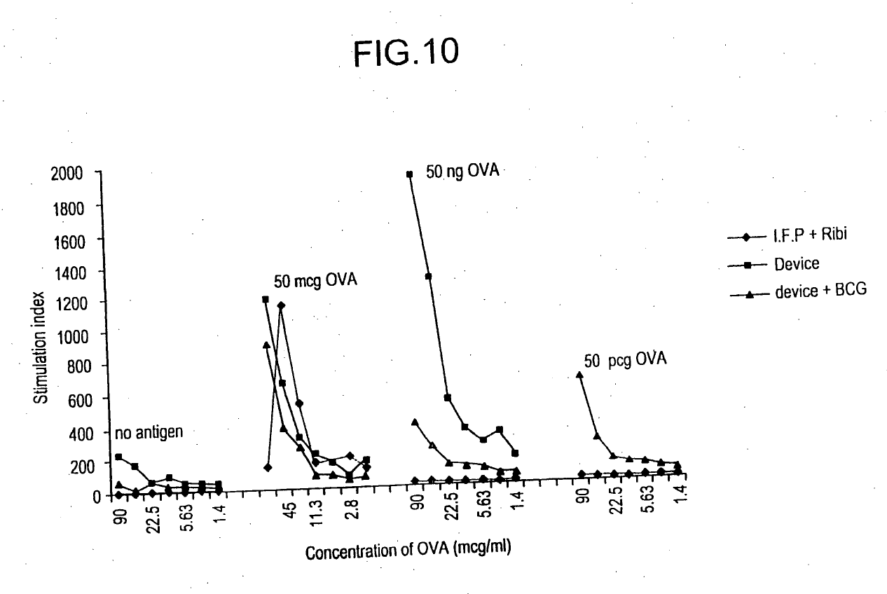

FIG. 10 shows that a robust antigen-specific T-cell response from low doses of antigen can be achieved using the device of the present invention. Negligible levels of stimulation were detected after spleen cells were exposed to the control antigen, indicating further that the response was specific to the immunogen.

### EXAMPLE 8

The antigen-specific antibody response against HIV gp120 peptide (residues 315-322, RIQRGPGRAFVTIGK) antigen was assessed after loading very low doses into the devices of the present invention. BALB/c mice were immunized either with the device or by footpad, with Ribi adjuvant, with various doses of HIV peptide. Blood was collected 10 days following immunization, and antibody titer against the peptide was determined. Four days later, the animals were boosted with 10% of the amount of the initial immunogen, and a second bleed was performed 10 days later for determination of antibody titer.

An ELISA was used to measure antigen-specific subclass IgG2a antibody, and was conducted according to a routine procedure. Briefly, microtiter plates were coated with the antigen (1 g/ml) in phosphate-buffered saline, for 16 hrs at 4° C. The plates were washed (50 mM Tris+0.2% Tween-20 in PBS, pH 7-7.5) and blocked with blocking buffer (5% BSA solution+0.1% Tween-20 in PBS, pH 7.2-7.4) at 4° C. for 16 hrs. The plates are washed and 100 l of serum samples are added to the wells starting from 1:50 dilution and further diluted with 3 fold steps. The samples are tested in duplicate. Following 1 hr of incubation at room temperature, the plates are washed as above and biotinylated anti-mouse IgG2a antibody solution (1 μg/ml) is added to the wells. Following 1-hour incubation and extensive washes as above streptavidin-conjugated horseradish peroxidase, diluted to 1:4000 in blocking buffer, is added to the wells. Following 30 minutes incubation at room temperature, substrate is added (100 l/well of tetramethylbenzidine). The plates are incubated for 30 minutes. Adding 100 l/well of 2 N H₂SO₄ stops the reaction. The plates are read using ELISA Plate Reader at wavelength of 450 nm.

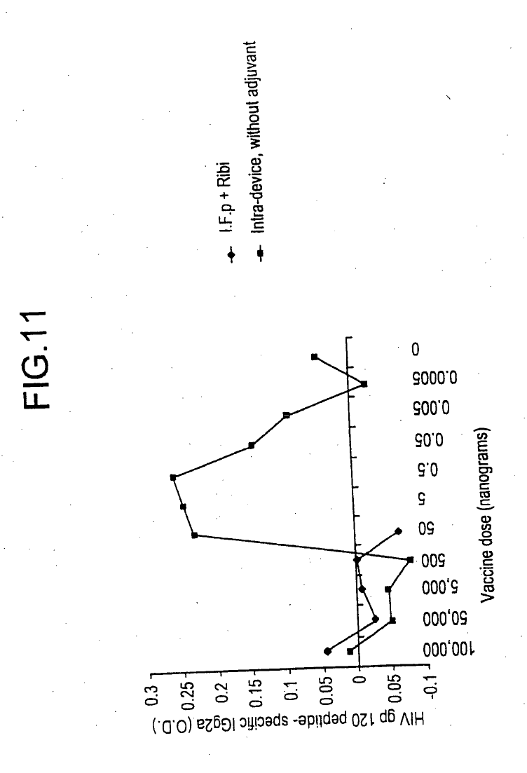

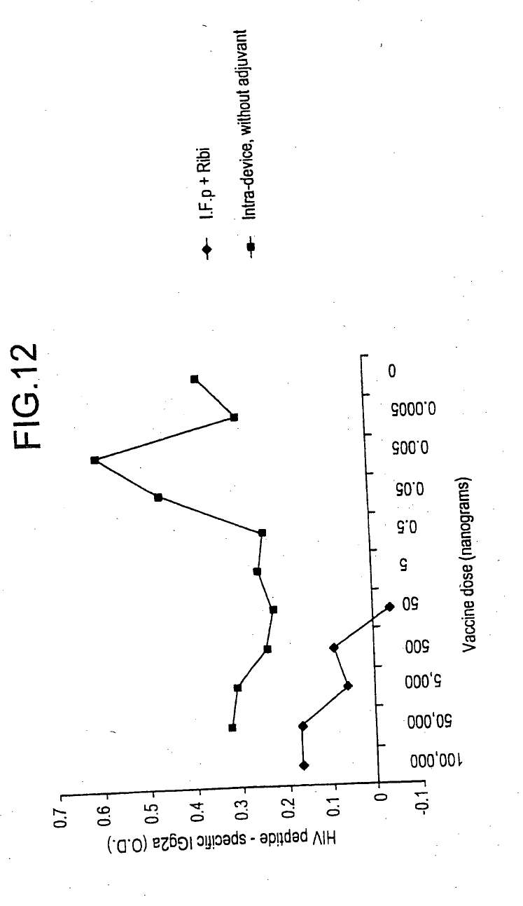

FIG. 11 shows the level of specific anti-HIV gp120 peptide IgG2a and indicates that following a single immunization using the device of the present invention, a strong antigen-specific antibody (IgG2a) is elicited, whereas no response was elicited by a single footpad immunization following a conventional protocol. Following a second immunization (boost) in the footpad, an antibody response was generated in animals immunized conventionally, but the animals initially immunized using the device of the present invention and then boosted (with 10% of the original dose administered within the device) showed increased IgG2a levels and still a significantly more robust response at the lower antigen levels (FIG. 12).

### EXAMPLE 9

The effect of different adjuvants was evaluated on the resulting immune responses generated from immunization with the device of the present invention compared with conventional footpad immunization. BALB/c mice were immunized with either the device or via footpad, with the HIV gp120 peptide (of the previous example) at various doses either alone or with Ribi or BCG adjuvants, as described above. The device was provided with immunogen three days after implantation, and antibody titers were determined 10 days post-immunization. An ELISA to IgG2a antibody specific for the gp120 peptide antigen was performed as described in the prior example.

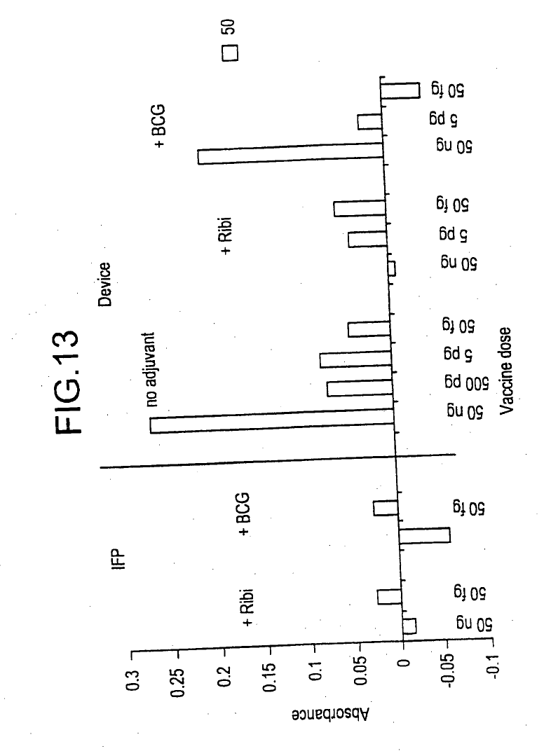

Conventional immunization with Ribi or BCG adjuvants produced a low level of peptide-specific IgG2a (FIG. 13). In contrast, immunization using the device of the present invention without adjuvant (50 ng peptide) produced a markedly high level of peptide-specific IgG2a. The peptide in combination with the either adjuvant (Ribi or BCG) produced a low response. These data imply that addition of an adjuvant to an already strong antigenic stimulus results in depression of the immune response. It is possible that in order to demonstrate the additive effect or synergism of adjuvant and antigen, immunization protocols should be tested at lower ranges (lower than femtogram doses) of antigen as well as lower concentrations of adjuvants.

Similar results were obtained when a 15-mer peptide of the Herpes Simplex virus glycoprotein B (residues 497-507: TSSIEFARLQF) was tested.

### EXAMPLE 10

In a further test of the strength of the humoral immune response induced by an antigen present in the device of the present invention, BALB/c mice were implanted with devices which were provided three days later with various doses of cytochrome C. As controls, various doses of the same antigen were administered via footpad in combination with Ribi adjuvant. Ten days post implantation or ten days post footpad inoculation, the animals were bled and an ELISA performed on serum for IgG2a that specifically recognizes the antigen. Procedures were similar to that followed in the previous example, except that the ELISA was performed on serial dilutions of the serum, from 1:50 to 1:6400.

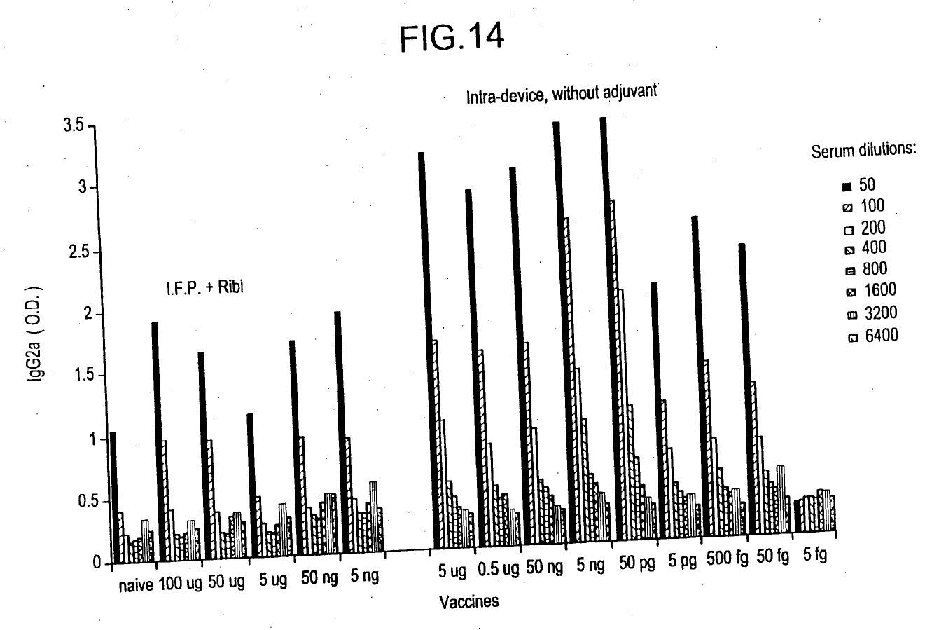

FIG. 14 shows that a very strong specific humoral immune response to the antigen was achieved with very low doses of antigen used in the device of the present invention. As low as 50 femtograms of antigen in the device yielded an antibody response similar to that achieved by conventional footpad plus adjuvant immunization using 5 ng antigen, an increase of 4 orders of magnitude.

### EXAMPLE 11

The development of a humoral response was further evaluated using as antigen the hemagglutinin protein, cleaved and purified from Influenza A virus (broken hemagglutinin antigen, or BHA). BALB/c mice were immunized using the device of the present invention with 0.1 μg and 10 μg doses, provided in the device 3 days post-implantation. Control mice were immunized with the same amount of antigen subcutaneously at the base of the tail, with Ribi adjuvant. The mice were bled on day 7, 14, 21 and 28 post-immunization and an ELISA performed on the serum for antigen-specific total IgG antibody.

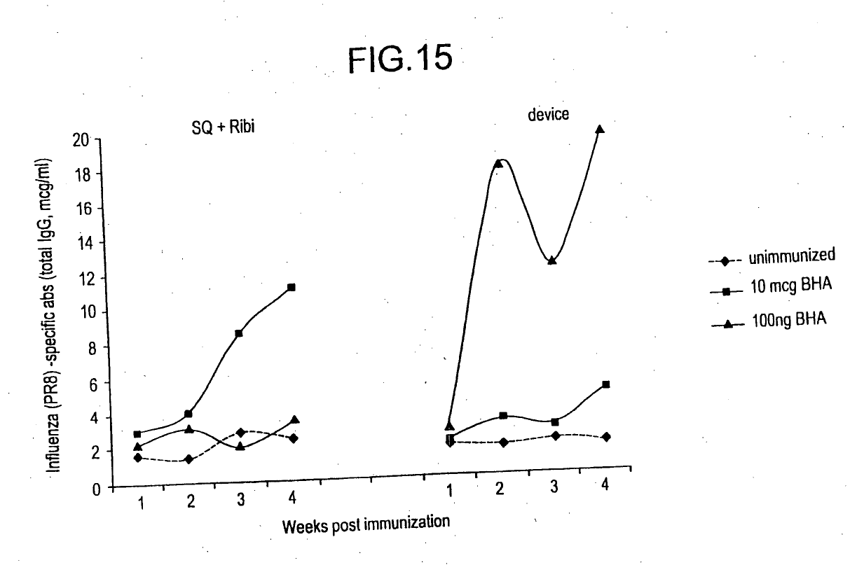

As shown in FIG. 15, a strong humoral response was obtained with the 0.1 μg antigen, reaching high concentration of HA-specific antibodies as soon as 2 weeks post-immunization. Mice immunized using the conventional protocol with adjuvant exhibited a low level of antigen-specific humoral response at the second week, increasing its level 3 and 4 weeks post-immunization. The level of BHA-specific antibody in serum of mice immunized with the device was 75-80% higher than the level achieved following conventional immunization with higher dose of antigen and in the presence of adjuvant. In this experiment, the low dose immunogen in the device produced a superior humoral response than was achieved with a higher dose. As seen in previous experiments, doses greater than 5 μg can result in suppression of the immune response possibly due to provocation of apoptosis as a reaction to overstimulation.

### EXAMPLE 12

The importance of the perforated but otherwise impermeable and biologically inert coating or container provided around the sponge matrix of the device of the present invention was evaluated by immunizing mice with influenza antigen either within the intact device as described above, or within the sponge matrix alone, implanted beside a segment of perforated tubing. A range of antigen doses were evaluated (0, 50 pg, 500 pg, 5 μg, and 50 μg) and were provided into the devices, or into the sponges alone, three days after implantation. As a control, the same ranges of antigen were immunized in the footpads of mice, together with Ribi adjuvant. Ten days after implantation or footpad immunization, spleens were harvested from the animals and a T-cell proliferation assay was performed as described above, using a range of antigen levels from 0.1 to 180 μg/ml. As a control for specificity, Epstein-Barr virus was used in vitro. ³H-thymidine incorporation was expressed as a Stimulation Index, to represent T-cell stimulation and proliferation.

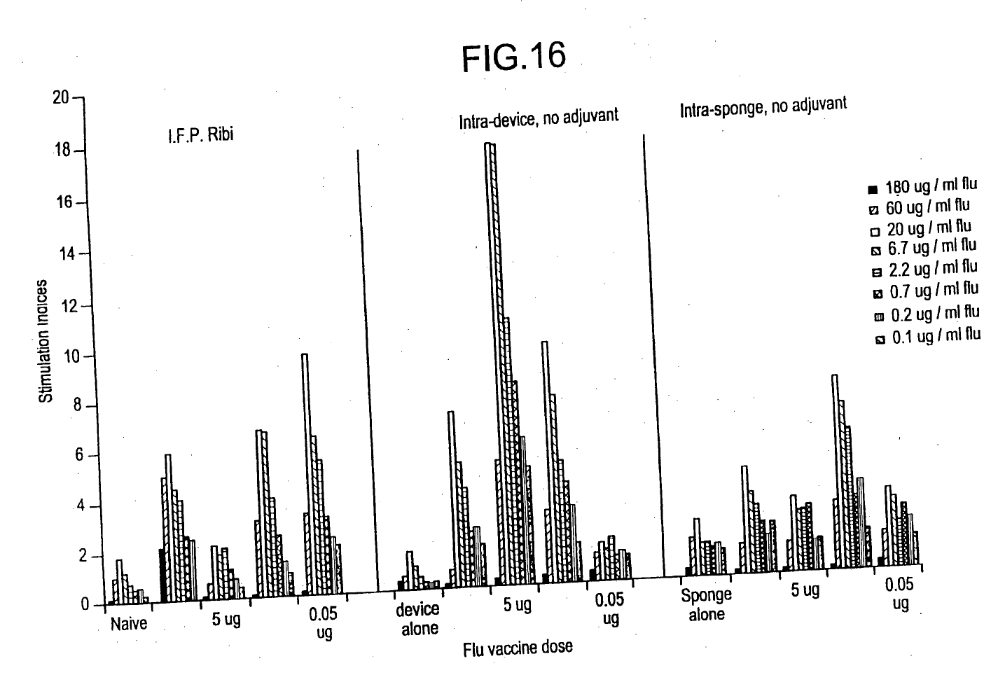

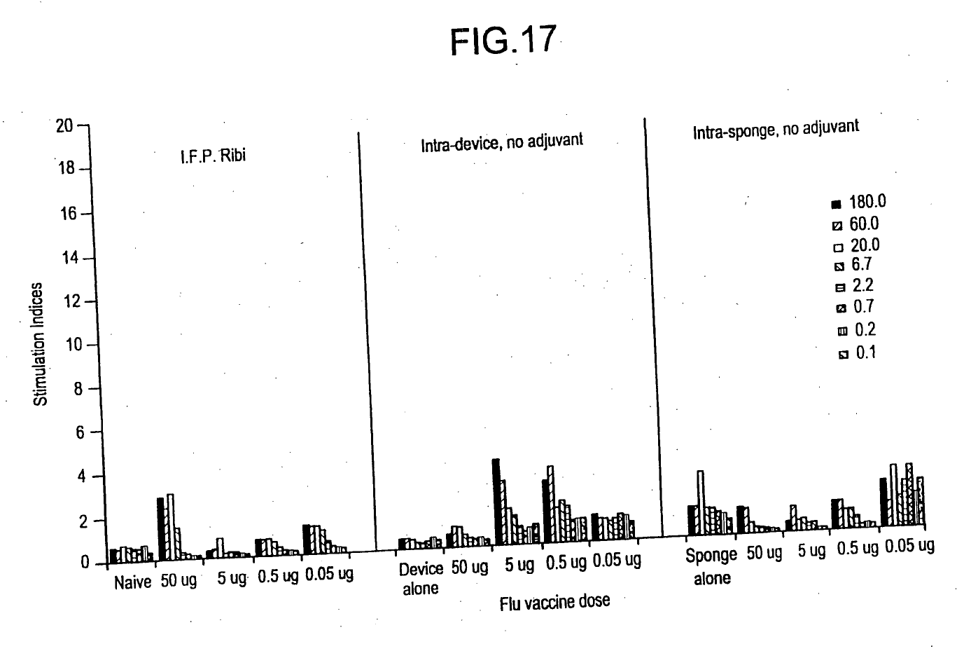

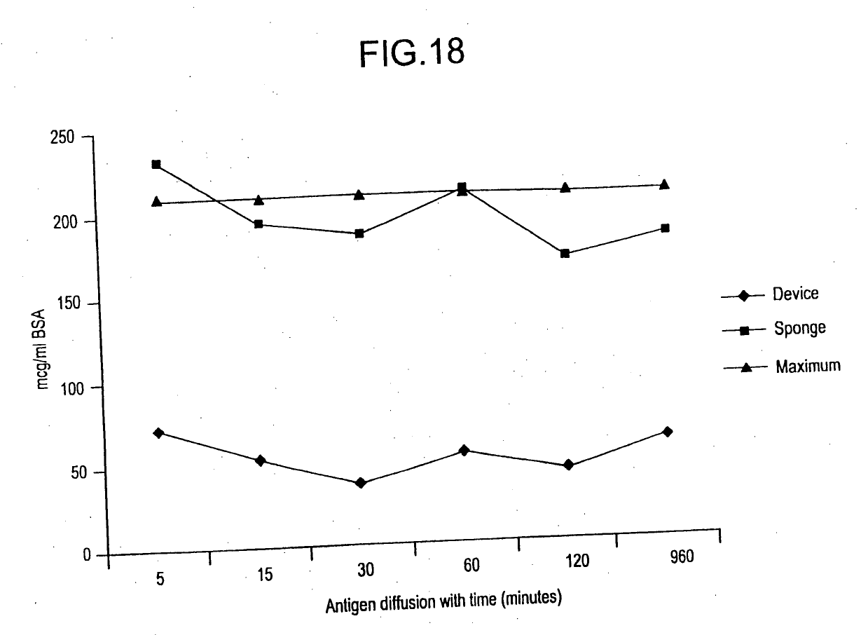

As shown in FIG. 16, T-cell activation exhibited by the animals immunized with the intact device showed a significantly stronger response than when the antigen was present in sponges not present within the perforated but otherwise impermeable container. The response to the unrelated antigen, FIG. 18, was minimal.

### EXAMPLE 13

The importance of the perforated but otherwise impermeable and biologically inert coating or container provided around the sponge matrix of the device of the present invention was evaluated by performing a diffusion test. Two hundred μg of bovine serum albumin (BSA) was injected into the device or into a sponge. The device and the sponge were placed into tubes containing 1.5 ml of PBS. Samples were taken at various time points and the BSA concentration was measured.

As shown in FIG. 18, the sponge released its entire content within 5 minutes of incubation. The device of the present invention controlled the release of the antigen and over 65% of the antigen was retained for over 960 min. Maintenance of a diffusion barrier to sustain high levels of antigen, cytokines, chemokines, and other co-stimulatory molecules within the device in contact with immune cells is postulated to be one of the important mechanisms providing the significant improvement of the device over conventional immunization procedures in provoking a robust immune response.

Thus, the examples presented above demonstrate the ability of the device of the present invention to stimulate a superior T cell and humoral response to a variety of antigens, in comparison to conventional footpad immunization and in comparison to simply loading antigen into an implanted sponge matrix.

### EXAMPLE 14

The phenotypes of cells present in the device of the present invention without added antigen were determined after implantation. Cells extracted from the device of the present invention at 4 days post implantation were collected, washed, and stained with fluorescein isothiocyanate- or phycoerythrin-labeled monoclonal antibodies specific to the following markers: CD14, CD45/B220, CD11b, CD40, CD11c, CD80, CD86, CD3, and I-Ad (PharMingen, Calif.). Geometric means of the fluorescence were determined by flow cytometry. For comparison, the fluorescence of peripheral blood lymphocytes (PBL) from naive, syngeneic mice using the same array of specific antibodies was determined, and the results for each antibody expressed as mean fluorescence intensity (MFI).

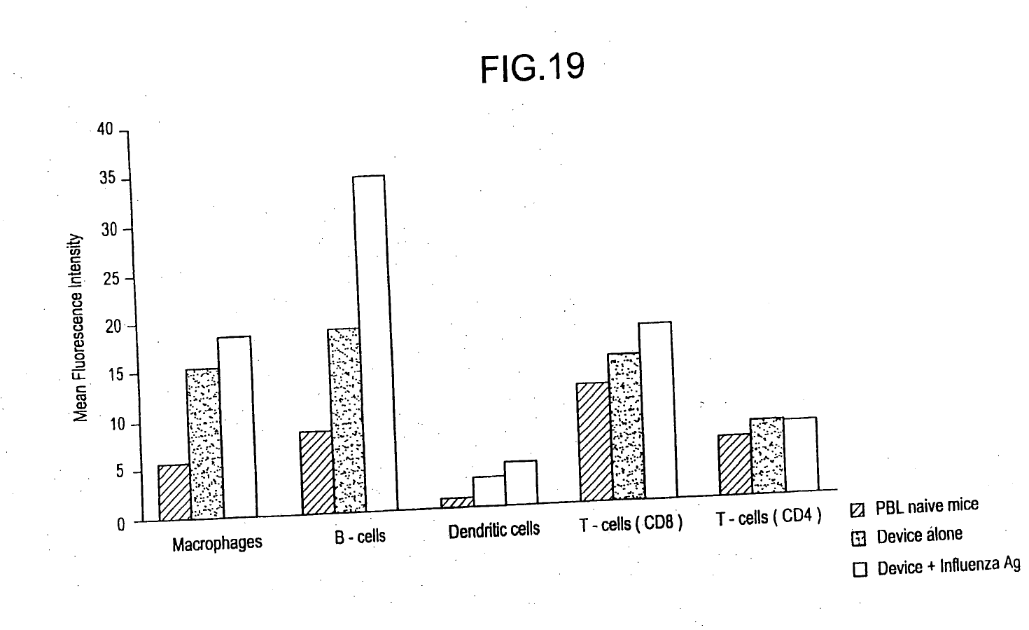

FIG. 19 presents the phenotype of cells extracted from the device 4 days post insertion. Migration and accumulation of T cells (CD3, CD40), macrophages (CD14, CD11b, class II MHC, CD80), dendritic cells (CD40, CD11c, Class II MHC, CD80), and B cells (Class II MHC, CD45/B220, CD11b) in high density were evident in the device. Thus, immune cells critical for the development of the immune response are present in the device. The expression of co-stimulatory (CD80 & CD86) and adhesion (CD44) molecules was significantly elevated on immune cells extracted from the device of the present invention comparative to PBL (data not shown). Interestingly, electron microscopy analysis of sections of the device of the present invention revealed the presence of macrophages with large numbers of lysosomes, implying their heightened state of cell activation.

### EXAMPLE 15

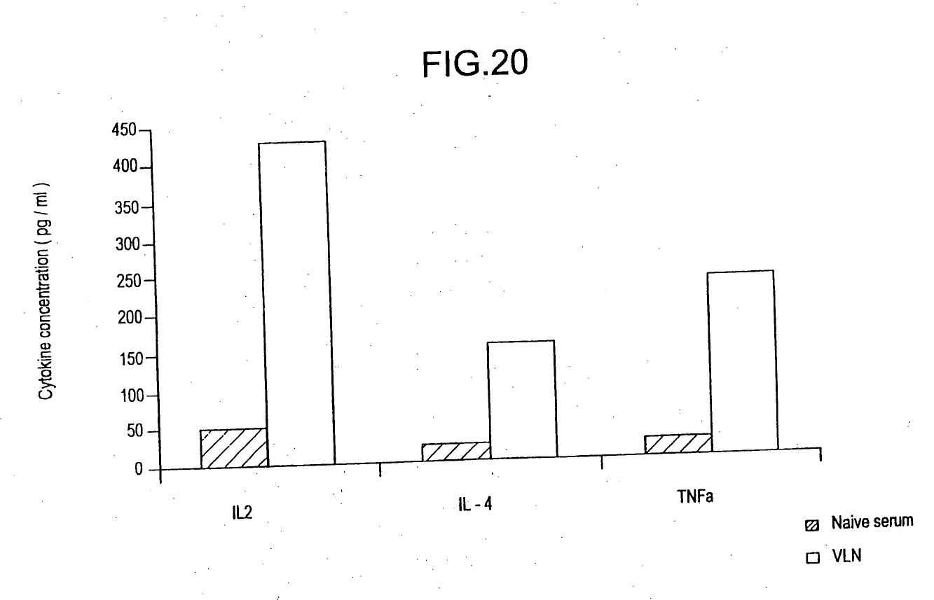

After implantation, the concentration of cytokines contained within the device of the present invention in the absence of antigen was compared with the concentration of cytokines in native murine serum. As shown in FIG. 20, the device contained greater levels of the stimulatory cytokines IL-2, IL-4, and TNF-α and approximately one-seventh the concentration of the inhibitory cytokine TGF-β (data not shown). Thus, the device of the present invention is a highly charged environment poised for eruption of immune response once “ignited” with an antigen.

### EXAMPLE 16

Cytotoxic T lymphocyte (CTL) responses are believed to be critical for the development of immunity to influenza virus. To evaluate the ability of the device of the present invention to elicit antigen-specific CTLs, mice were immunized with 500 pg of influenza virus vaccine in the device of the present invention. As a control, mice were immununized subcutaneously with 500 pg vaccine plus Ribi adjuvant. Lymphocytes were obtained from the immunized animals and their specificity for influenza virus was determined by lysis of influenza virus-infected target cells.

CTLs were prepared as follows. Mice were euthanized and the spleens were removed. Single cell suspensions were prepared and the cells were washed with PBS. Red blood cells were depleted using Red Cell Lysis Buffer (Sigma, Mo.). Spleen cells (5×10⁶) were co-cultured in vitro with 5×10⁶ virus infected (100 HAU/10⁷ cells/ml for 60 minutes at 37 C) and irradiated (3000Rad) syngeneic spleen cells and incubated for 5 days at 37 C, 5% CO₂. PR8 infected (500 HAU/10⁶ cells) P1.HTR cells and non-infected P1.HTR cells were used as targets for cytotoxicity. The cells were labeled with 100 mCi of Na₂⁵¹CrO₄ for 90 minutes and then washed three times. Target cells (10⁴/ml) in 100 ml of RPMI 1640 and 5% FCS medium were added to effector cells in various concentrations starting at a 100:1 ratio (E:T) and titrating two-fold in 96-well round bottom microtiter plates. After a 5-hour incubation, supernatant was harvested. Results are expressed as a percentage of specific ⁵¹Cr release according to the formula:

[(Experimental release)−(Spontaneous release)]×100/[(Maximum Release)−(Spontaneous Release)].

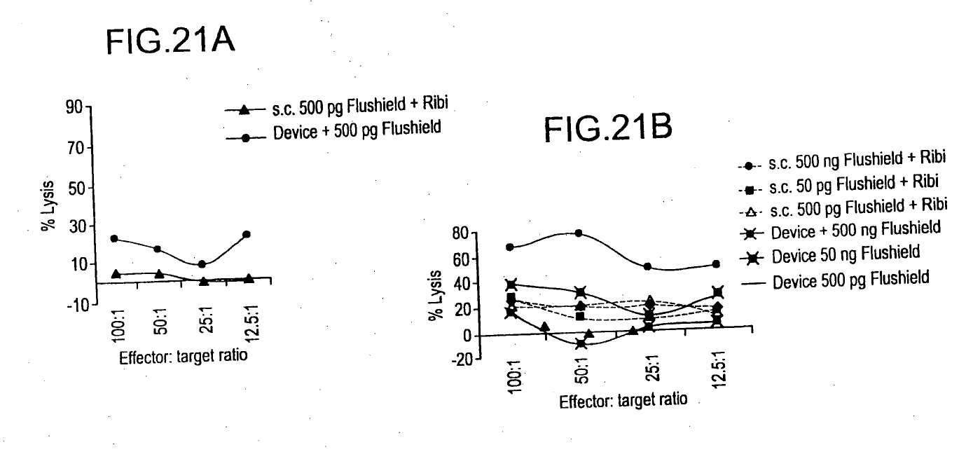

As shown in FIG. 21, immunization using the device of the present invention with 500 pg of influenza virus vaccine triggered powerful influenza-specific CTLs that were significantly better at lysing influenza-infected target cells, compared to CTLs produced in response to conventional subcutaneous immunization with Ribi adjuvant. The generated CTL did not kill non-infected target cells in vitro, indicating the specificity of the provoked response. With higher doses of the antigen in the device of the present invention, no stimulation was seen.

### EXAMPLE 17

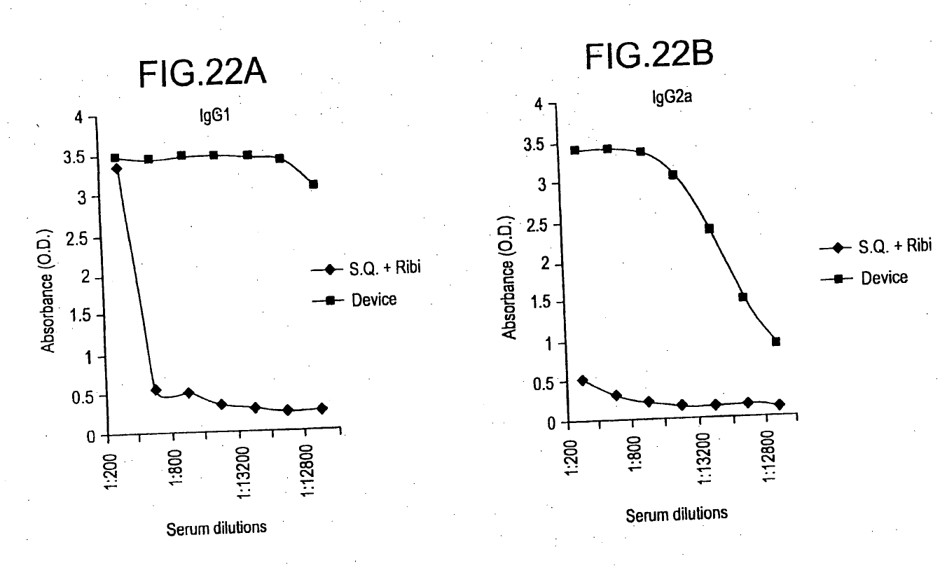

To determine that immunization with the device of the present invention could also modulate the humoral immune response, mice were immunized with several distinct doses of influenza virus vaccine either administered in the device of the present invention or injected subcutaneously with Ribi adjuvant. High levels of influenza-specific IgM, IgG1, and IgG2a antibodies were demonstrated in sera of mice following immunization with the device of the present invention with lower doses of vaccine as compared with subcutaneous immunization in combination with Ribi (data not shown). Examination of the virus-specific IgG1 antibodies revealed higher binding as measured by the absorbance recorded at escalating serum dilutions (FIG. 22). Similar results were observed for IgG2a antibodies.

### EXAMPLE 18

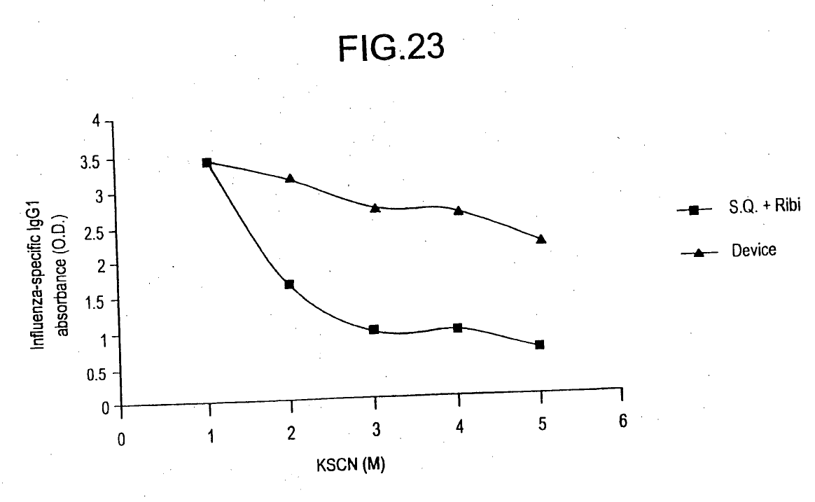

Serum antibodies generated by immunization using the device of the present invention relative to the conventional protocol with adjuvant were subjected to a gradient of escalating molarity of KSCN (potassium thiocyanate) used to elute antigen-specific antibodies from complexes bound to antigen-coated microtiter plates in order to determine the relative affinity of the antibodies to the influenza antigen. A standard microtiter plate-based ELISA format was used, as described above. Prior to adding biotinylated anti-mouse IgG, the plates were washed and the various concentrations of KSCN were added to the wells. Plates were incubated at room temperature for 30 minutes. After washing, biotinylated anti-mouse IgG1 antibody solution (0.5 μg/ml) was added to the wells. Increased concentrations of KSCN are necessary to break complexes created by the binding of high affinity antibodies to antigen. As depicted in FIG. 23, the developed antibodies were shown to possess higher affinity to the viral antigens. Thus, a very small dose of vaccine is sufficient to stimulate a powerful humoral response when using the device of the present invention.

### EXAMPLE 19

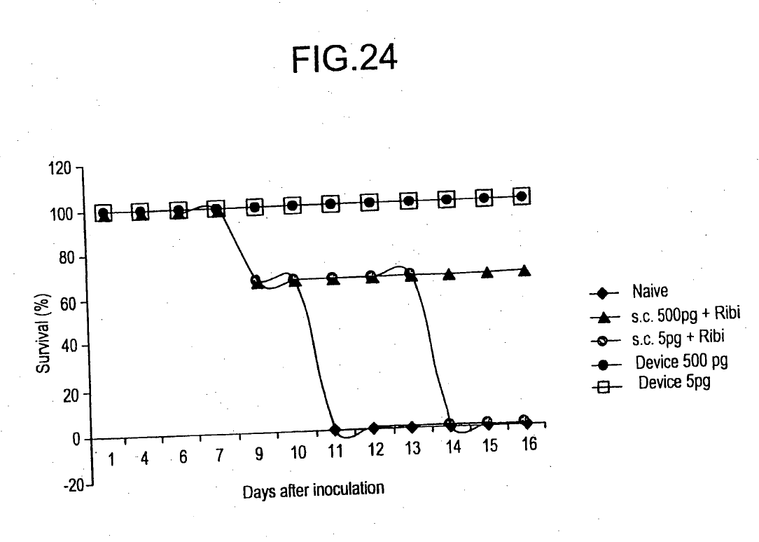

The capacity of immunization using a device of the present invention to protect mammals against infectious diseases was tested in a murine influenza model. Two doses of influenza virus vaccine influenza vaccine (5 pg and 500 pg) were used to immunize mice with either the device of the present invention or by subcutaneous administration in combination with Ribi adjuvant. Approximately 12 weeks after vaccination, animals were challenged with a lethal dose of influenza virus. As shown in FIG. 24, vaccination using a device of the present invention with both the 5 pg and 500 pg doses of influenza vaccine resulted in a complete protection, whereas only 60% of the mice immunized subcutaneously with 500 pg of influenza vaccine plus Ribi adjuvant survived. All of the animals immnunized with the 5 pg dose plus adjuvant succumbed to the disease by day 14. Remarkably, not only did immunization using a device of the present invention provide total protection, but it was also accomplished using one-hundredth the amount of antigen and no adjuvant. Mice immunized with the device of the present invention also exhibited steady weight following the viral challenge, indicating development of a milder disease correlated with their relative resistance to acute infection.

### EXAMPLE 20

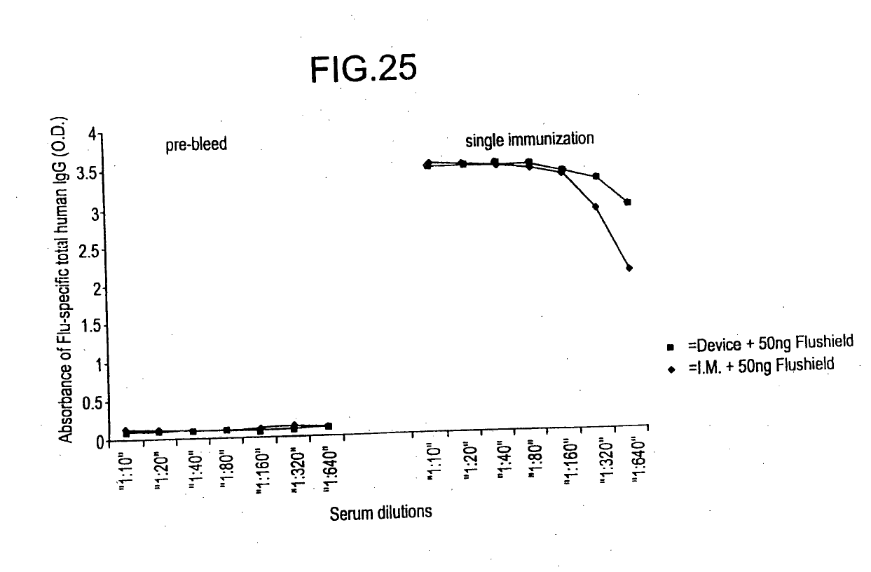

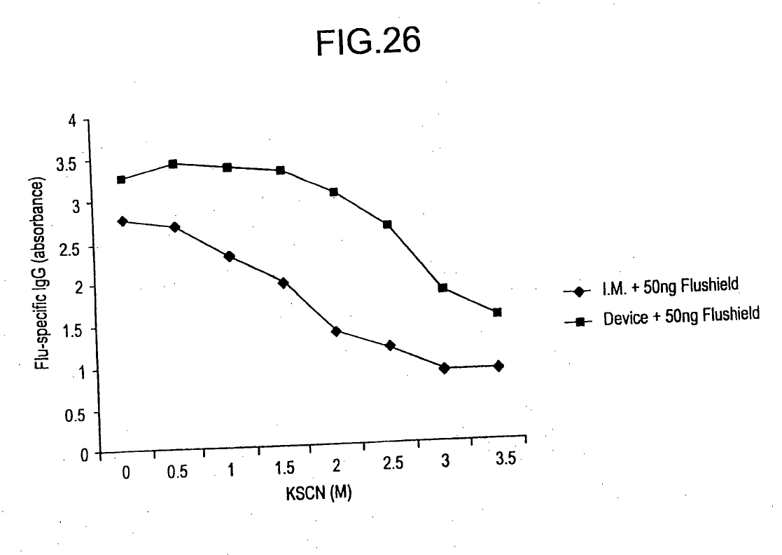

The utility of the device of the present invention for generating specific human IgG in severe combined immunodeficiency (SCID) mice populated with human immune cells was evaluated by implanting a device containing influenza virus vaccine. SCID Beige CD17 (6-8 weeks old female) mice were infused (intraperitoneally) with 20×10⁶ PBMC from a normal human donor. Three days later the mice were immunized either intramuscularly or implanted with a device of the present invention pre-pulsed with 50 ng influenza virus vaccine. Three weeks post-immunization the mice were bled and the sera was tested for influenza-specific humoral response, using conventional ELISA. As shown in FIG. 25, immunization with the vaccine generated influenza-specific human IgG. Furthermore, as shown using KSCN at escalating molarity to measure the relative affinity of the serum antibodies to influenza antigen (as described above in Example 18), the device of the present invention produced a higher titer human influenza-specific antibody than that achieved by traditional, intramuscular immunization (FIG. 26). This method can also be used to generate human hybridomas capable of producing human monoclonal antibodies.

### EXAMPLE 21

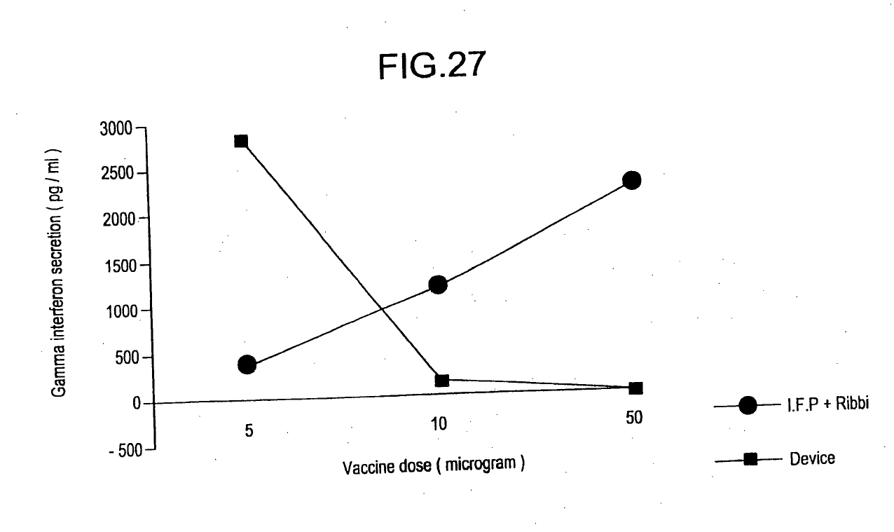

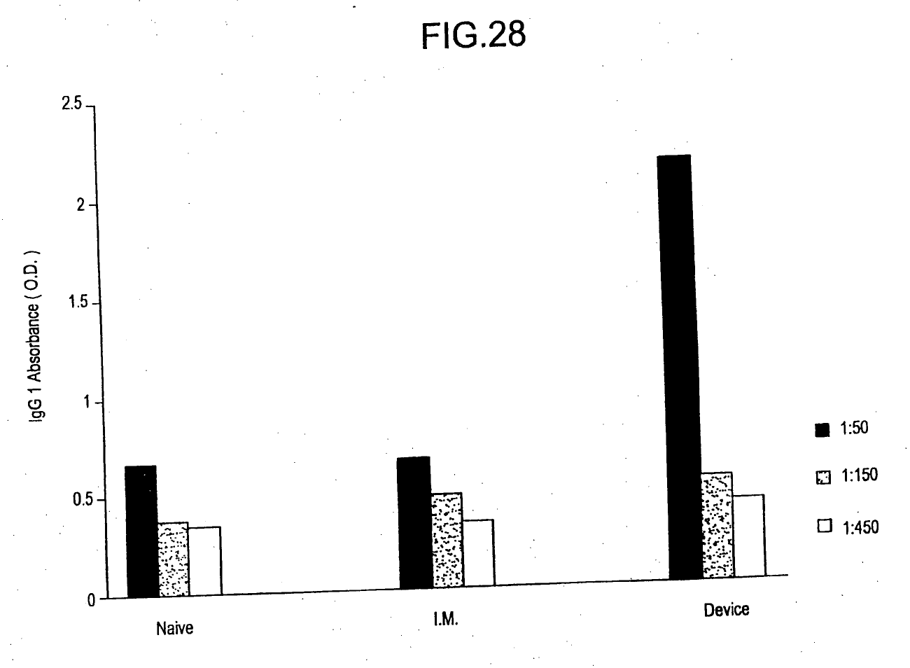

Higher amounts of antigen provided in a device of the present invention can cause a suppression of the immune response. Balb/c mice were implanted with the device loaded with the 5 to 50 μg of influenza vaccine or immunized in the intra-footpad with the antigen mixed with Ribi adjuvant. Mice were euthanized ten days post-immunization and spleen cells were isolated and plated in 96 well plates (2×10⁵ cells/well). Cells were exposed to influenza antigen for 72 hours at 37 C. Following stimulation with antigen, supernatants were removed from the cultures and gamma-interferon production was measured using an ELISA kit (Endogen). As shown in FIG. 27, the mice immunized with a combination of the antigen and the Ribi adjuvant gave an immune response which increased directly with the dose of antigen. In contrast, the mice immunized with antigen inside the device showed a stimulation of the immune response at the low doses of antigen (as noted in previous Examples herein) but a decrease in the immune response at the higher doses of antigen. Thus, the presence of a large amounts of antigen in an implanted device of the present invention leads to a down regulation of the immune response. This method can be used in treatment strategies where specific immunosuppression is useful, e.g., transplantation, allergy, etc.

### EXAMPLE 22

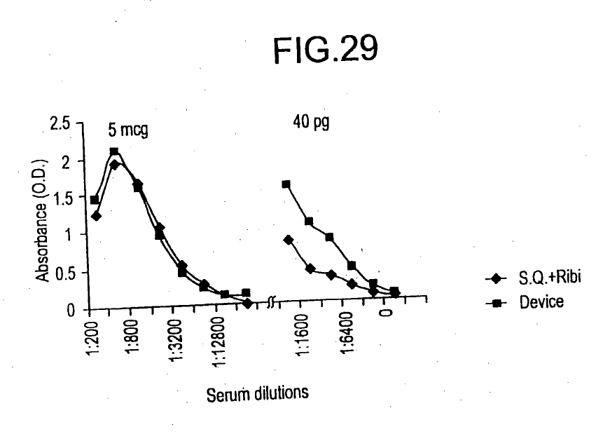

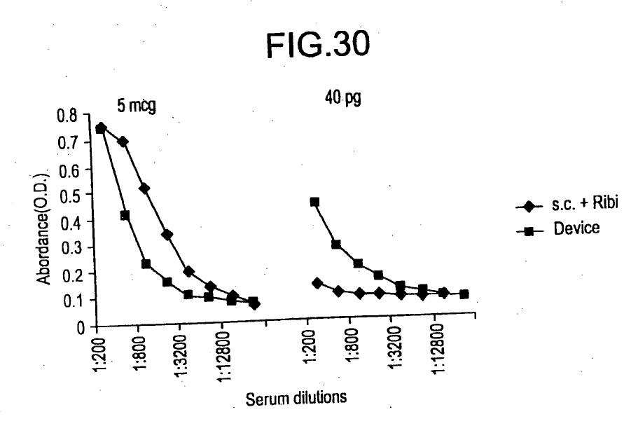

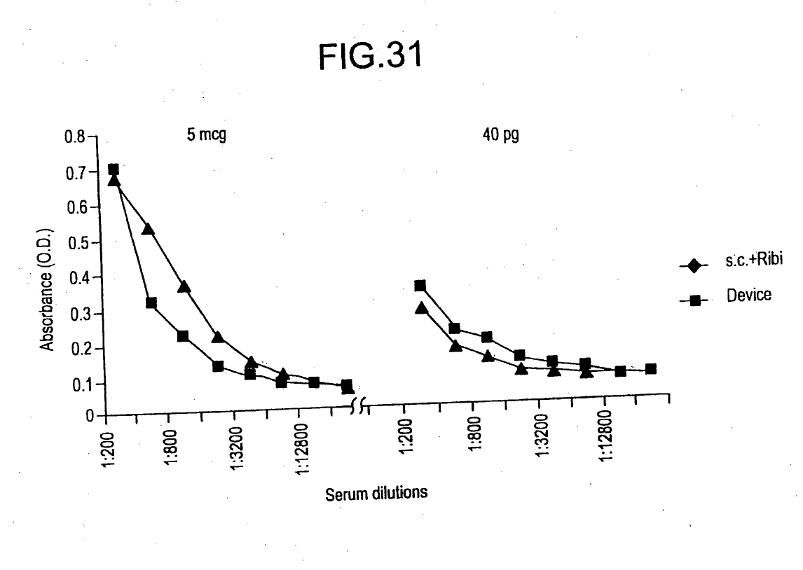

The device of the present invention can also be used to generate an immune response to a polysaccharide antigen. A single immunization using a device loaded with pneumococcal vaccine (Pneumovax-23) produces a specific immune response. Pneumovax is a polyvalent pneumococcal vaccine consisting of highly purified capsular polysaccharides from the 23 most prevalent or invasive pneumococcal types. Mice were immunized with either 5 μg or 40 pg of the Pneomovax vaccine either in a device implanted subcutaneously as described above, or mixed with Ribi adjuvant and injected into the footpad. All mice were bled on Day 10 and assayed by ELISA for total IgG (FIG. 29), IgG1 (FIG. 30) and IgG2a (FIG. 31) response. These results show that an immune response to polysaccharide antigens can be achieved using the device of the present invention.

### EXAMPLE 23

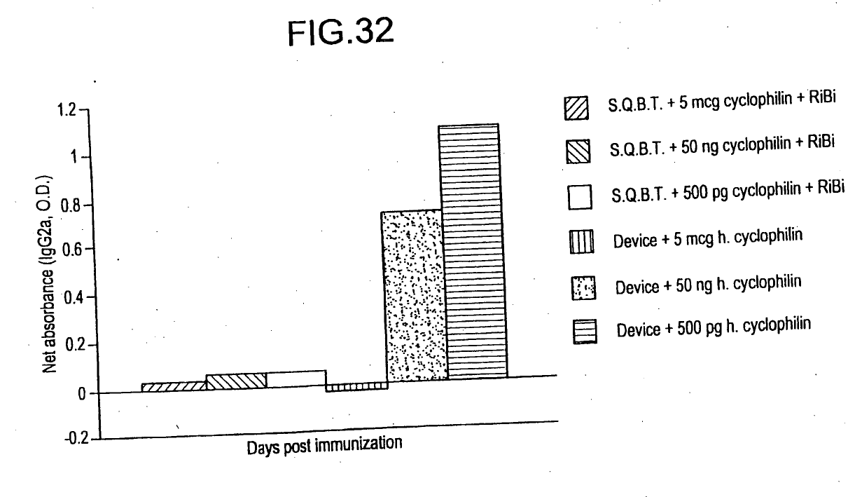

The device of the present invention can be used to generate an immune response to a highly conserved antigen. Cyclophilin was selected as a model protein for these experiments, since development of an immune response to cyclophilin has been difficult to achieve, since this protein is highly conserved among mammalian species. Mice were immunized with 5 μg, 50 ng or 0.5 ng of human cyclophilin either loaded into a device of the present invention and implanted subcutaneously, or mixed with Ribi adjuvant and injected subcutaneously. Mice were bled and the sera was tested for an IgG2a response to human cyclophilin by ELISA. As shown in FIG. 32, mice immunized with the two lower doses of antigen using the device of the present invention showed a superior response to the antigen in comparison with the mice immunized subcutaneously with cyclophilin plus Ribi adjuvant. This result demonstrates that the device can induce an immune response to highly conserved antigens which are otherwise non-immunogenic. Immunization with the high dose of antigen in the device of the present invention fails to induce an immune response.

### EXAMPLE 24

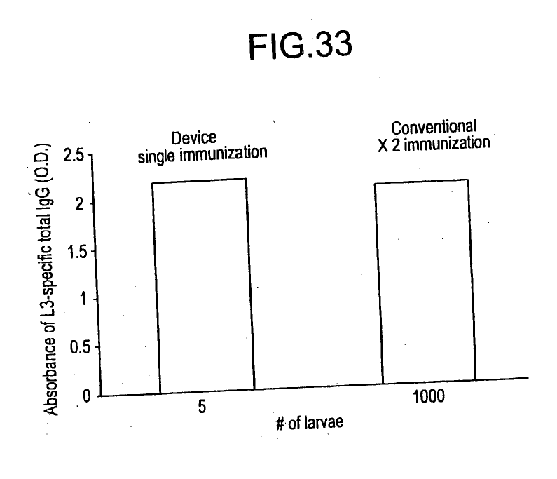

The device of the present invention can be used to generate an immune response to whole, live organisms. In this experiment, mice were immunized with live hook-worm larvae using either 5 larvae provided inside a subcutaneously-implanted device of the present invention, or using 1000 live larvae injected subcutaneously 2 weeks apart. As shown in FIG. 33, both protocols induced L3-specific total IgG as measured by ELISA. It has been possible to immune against a number of parasitic organisms including hookworm and malaria using irradiated larvae and sporozoites, respectively; studies herein indicate that immunization using the device of the present invention can be achieved using a decreased number of organisms.

This invention may be embodied in other forms or carried out in other ways without departing from the spirit or essential characteristics thereof. The present disclosure is therefore to be considered as in all respects illustrative and not restrictive, the scope of the invention being indicated by the appended Claims, and all changes which come within the meaning and range of equivalency are intended to be embraced therein.

Various publications are cited herein, the disclosures of which are incorporated by reference in their entireties.

What is claimed is:

1. A method for modulating the immune response in a mammal to an antigen by implanting within the body of said mammal a device comprising a porous matrix contained within a perforated but otherwise impermeable container, said matrix containing a quantity of said antigen, wherein said device attracts cells of the immune system to encounter said antigen and to modulate said immune response.

2. The method of claim 1 wherein the antigen is bioavailable within said porous matrix at the time of implantation of said device into said mammal.

3. The method of claim 1 wherein the antigen becomes bioavailable within said porous matrix after the device has been implanted into said mammal.

4. The method of claim 3 wherein said antigen becomes bioavailable about three days after implantation within said mammal.

5. The method of claim 1 wherein said antigen is introduced into said device about three days after implantation.

6. The method of claim 1 wherein said antigen is provided in a delayed release formulation.

7. The method of claim 1 wherein said porous matrix comprises a polymeric material.

8. The method of claim 7 wherein said polymeric material is selected from natural and synthetic sources.

9. The method of claim 8 wherein said polymeric matrix is selected from the group consisting of hydroxylated polyvinyl acetate, polyurethane, ethylene/vinyl acetate copolymer, polylactic acid, polylactide-glycolide copolymer, gelatin, collagen, cross-linked collagen, and combinations thereof.

10. The method of claim 1 wherein said container comprises a polymeric material selected from natural and synthetic sources.

11. The method of claim 1 wherein the porous polymer matrix comprises hydroxylated polyvinyl acetate and the container comprises a segment of perforated tubing.

12. The method of claim 1 wherein said quantity of antigen and the timing of the bioavailability of said antigen within said device relative to the time of implantation of said device into said mammal results in inducing or enhancing the immune response to said antigen.

13. The method of claim 12 wherein said antigen is bioavailable within said device after implantation of said device into said mammal.

14. The method of claim 13 wherein said antigen is introduced into said device about 2-4 days after the implantation of said device into said mammal.

15. The method of claim 1 wherein said quantity of antigen and the timing of the bioavailability of said antigen within said device relative to the time of implantation of said device into said mammal results in suppressing or down regulating an existing or potential immune response to said antigen.

16. The method of claim 15 wherein said antigen is bioavailable within said device at the time of implantation within said mammal.

17. The method of claim 1 wherein said device is removed from the body of said mammal after a period of about 10 days.

18. The method of claim 1 wherein a second quantity of said antigen is reintroduced into said device.

19. The method of claim 18 wherein said second quantity of said antigen is reintroduced into said device by delayed release of said second quantity of said antigen present within the device at the time of implantation.

20. An implantable device for modulating an immune response to an antigen comprising a porous matrix contained within a perforated but otherwise impermeable container.

21. The device of claim 20 wherein said antigen is present within said porous matrix.

22. The device of claim 20 further comprising means for introducing said antigen into contact with said porous matrix, either prior to or after implantation.

23. The device of claim 20 wherein said matrix comprises a polymeric material.

24. The device of claim 23 wherein said polymeric material is selected from natural and synthetic sources.

25. The device of claim 24 wherein said polymeric material is selected from the group consisting of hydroxylated polyvinyl acetate, ethylene/vinyl acetate copolymer, polylactic acid, polylactide-glycolide copolymer, polyurethane, gelatin, collagen, cross-linked collagen and combinations thereof.

26. The device of claim 20 wherein said container comprises a segment of perforated tubing.

27. The device of claim 20 wherein said container comprises a perforated but otherwise impermeable coating disposed around said porous matrix.

28. The device of claim 27 wherein said coating comprises a polymeric material.

29. The device of claim 28 wherein said polymeric material is selected from natural and synthetic sources.

30. The device of claim 29 wherein said polymeric material is selected from the group consisting of cross-linked collagen, polylactic acid, polylactide-glycolide copolymer, polyethylene, silicone, latex resin, polystyrene, acrylic resin, polyvinylpyrrolidone, and combinations thereof.

31. The device of claim 20 wherein the porous matrix comprises hydroxylated polyvinyl acetate and the container comprises a segment of perforated tubing.

32. A method for obtaining immune cells from a mammal wherein said immune cells are harvested from a device implanted in said mammal comprising a porous matrix contained within a perforated but otherwise impermeable container.

33. The method of claim 32 wherein said harvested cells are reintroduced into said mammal.

34. The method of claim 33 wherein said harvested cells are cryopreserved before reintroduction into said mammal.

35. The method of claim 32 wherein an antigen is present within the porous matrix of said device.

36. The method of claim 35 wherein said immune cells are reintroduced into said mammal.

37. The method of claim 35 wherein said immune cells are reintroduced into said mammal after exposure to said antigen in vitro.

38. The device of claim 32 wherein said matrix comprises a polymeric material.

39. The device of claim 38 wherein said polymeric material is selected from natural and synthetic sources.

40. The device of claim 39 wherein said polymeric material is selected from the group consisting of hydroxylated polyvinyl acetate, ethylene/vinyl acetate copolymer, polylactic acid, polylactide-glycolide copolymer, polyurethane, gelatin, collagen, cross-linked collagen and combinations thereof.

41. The device of claim 32 wherein said container comprises a segment of perforated tubing.

42. The device of claim 32 wherein said container comprises a perforated but otherwise impermeable coating disposed around said porous matrix.

43. The device of claim 42 wherein said coating comprises a polymeric material.

44. The device of claim 43 wherein said polymeric material is selected from natural and synthetic sources.

45. The device of claim 44 wherein said polymeric material is selected from the group consisting of cross-linked collagen, polyethylene, silicone, latex resin, polystyrene, acrylic resin, polylactic acid, polylactide-glycolide copolymer, polyvinylpyrrolidone, and combinations thereof.

46. The device of claim 32 wherein the porous matrix comprises hydroxylated polyvinyl acetate and the container comprises a segment of perforated tubing.

47. The method of claim 35 wherein said immune cells are used for the preparation of a hybridoma for the production of a monoclonal antibody against said antigen.

48. A method of immunizing a mammal with an antigen for the preparation of a hybridoma for the production of a monoclonal antibody against said antigen, wherein the mammal is immunized using the method of claim 12.

49. A method of immunizing a mammal with an antigen for the preparation of a hybridoma for the production of a monoclonal antibody against said antigen, wherein the mammal is immunized using the device of claim 21.

50. The method of claim 12 wherein said immune response to said antigen is selected from the group consisting of prophylactic vaccination, therapeutic vaccination, cellular immunity, humoral immunity, mucosal immunity, long-term immunity, and combinations thereof.

51. A method for the production of hybridomas producing human monoclonal antibodies against a preselected antigen comprising the sequential steps of:
(a) introducing human peripheral blood lymphocytes into the circulation of a severe combined immunodeficient (SCID) mouse and allowing said lymphocytes to populate the immune system of said mouse;
(b) implanting in said mouse a device of claim 21, the antigen of said device comprising said preselected antigen;
(c) harvesting immune cells from said device;
(d) preparing hybridomas from B lymphocytes present in said harvested immune cells; and
(e) identifying by screening methodology those hybridomas that produce monoclonal antibodies that recognize said preselected antigen.

52. A method for transfecting immune cells of a mammal with genetic material comprising introducing said genetic material within the matrix of a device comprising a porous matrix contained within a perforated but otherwise impermeable container, said device implanted within the body of said mammal.

53. The method of claim 52 wherein said genetic material is selected from the group consisting of DNA, RNA, and cDNA.

54. The method of claim 52 wherein said genetic material codes for an antigen.

55. A method for the treatment or prophylaxis of a disease or condition caused by an immune response comprising suppressing said immune response in accordance with claim 15.

56. The method of claim 55 wherein said disease or condition is selected from the group consisting of allergies, transplant rejection, and autoimmune diseases.

57. A method for modulating the immune response in a mammal to an antigen by implanting within the body of said mammal a device comprising said antigen and further comprising means for limiting the passive diffusion of molecules out of said device without limiting the active movement of immune cells into or out of said device.
